(19) United States
(12) Patent Application Publication
(10) Pub. No.: US 2021/0327590 A1
(43) Pub. Date: Oct. 21, 2021

(54) RISK ASSESSMENT AND RISK REDUCTION IN TISSUE COLLECTION AND PROCESSING

(71) Applicant: Devicor Medical Products, Inc., Cincinnati, OH (US)

(72) Inventors: Adam Walter, Mason, OH (US); John Scott Ehlert, Cincinnati, OH (US)

(21) Appl. No.: 17/325,653

(22) Filed: May 20, 2021

Related U.S. Application Data
(63) Continuation of application No. PCT/US19/62412, filed on Nov. 20, 2019.
(60) Provisional application No. 62/770,324, filed on Nov. 21, 2018.

(51) Int. Cl. G16H 50/30; G16H 10/40; A61B 10/02

(52) U.S. Cl. CPC G16H 50/30; A61B 10/02; G16H 10/40

(57) ABSTRACT

A biopsy procedure may have ten or more high-level steps that a particular facility is able to perform, including radiology, transfer to pathology, grossing, processing and fixation, embedding, microtomy, H&E staining, coverslipping, slide preparation, and IHC. More than seven hundred individual risk points exist throughout the performance of each of these steps, each with varying likelihood of occurrence and severity of result. A software application provides a guided assessment that may be performed to assess a risk level for the facility. The assessment identifies approximately thirty observable characteristics of the facility, each of which may implicate one or more of the seven hundred risk points. Responses to the assessment may be used to produce a current risk metric for a facility, as well as identify a post-control risk metric for the facility, which may be achieved by implementing processes and controls to address the observable characteristics of the facility.

### PRIORITY

This application claims priority to U.S. Provisional Patent App. No. 62/770,324 entitled “Risk Assessment and Risk Reduction in Tissue Collection and Processing,” filed on Nov. 21, 2018, the disclosure of which is incorporated by reference herein.

### BACKGROUND

A biopsy is the removal of a tissue sample to examine tissue for signs of cancer or other disorders. Tissue samples are obtained in a variety of ways using various medical procedures involving a variety of the sample collection devices. For example, biopsies may be open (surgically removing tissue) or percutaneous (e.g. by fine needle aspiration, core needle biopsy or vacuum assisted biopsy). After the tissue sample is collected, the tissue sample is analyzed at a lab (e.g. a pathology lab, biomedical lab, etc.) that is set up to perform the appropriate tests (such as histological analysis).

Numerous steps, facilities, tools, and personnel are involved in a biopsy procedure. Some high volume biopsy providers may perform numerous procedures in the same day, using the same set of rooms and personnel, and tissue samples from each procedure may be collected and transported to an even more numerous set of destinations where they may be divided, prepared, diagnosed, and stored. With a desire for rapid turnover between procedures, facilities and personnel are under a constant stress to perform steps more quickly. In pursuit of efficiency, some facilities may develop or adopt certain time saving processes which may contribute to the risk of a misdiagnosis or other negative result from a biopsy procedure. For example, records from one or more upcoming patients may be kept in a stack and placed in a convenient location, which may reduce the time spent individually retrieving such records, but may increase the risk of patient records becoming mixed, which may cause a patient to receive the wrong type of procedure or treatment, or may cause a patient to receive a diagnosis intended for another patient.

It may be desirable, especially within such a setting, to identify and address such processes that unnecessarily contribute to the risk of a negative patient outcome. This can be difficult however, as these types of informal processes can become accepted within a certain facility or group of personnel over time, and so can be difficult to self-diagnose as a source of risk for patients. Similarly, an independent auditor may also have difficulty identifying such risks, as they may primarily rely upon interviewing personnel about such behavior and procedures. For example, when directly confronted with a line of questioning from an outside auditor, personnel may feel a pressure to omit information that they fear reflect poorly upon their facility or coworkers, and so the use of non-standard time saving practices may be left out of an interview in favor of more official or rigorous practices.

While several systems and methods have been made and used for obtaining and processing a biopsy sample, it is believed that no one prior to the inventor has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts an exemplary biopsy suite;

FIG. 2 depicts a flowchart of an exemplary general workflow that may be associated with a biopsy process;

FIG. 3 depicts a schematic diagram of an exemplary risk assessment system;

FIG. 4 depicts a schematic diagram of an exemplary audit device of the risk assessment system;

FIG. 5 depicts a flowchart of an exemplary set of high level steps that may be performed to provide a risk assessment;

FIG. 6 depicts a flowchart of an exemplary set of steps that may be performed to configure the audit device to assess a site;

FIG. 7 depicts a flowchart of an exemplary set of steps that may be performed to prepare an assessment for the site;

FIG. 8 depicts a flowchart of an exemplary set of steps that may be performed to receive assessment responses; and

FIG. 9 depicts a flowchart of an exemplary set of steps that may be performed to provide a set of assessment results.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

I. Exemplary Biopsy Suite

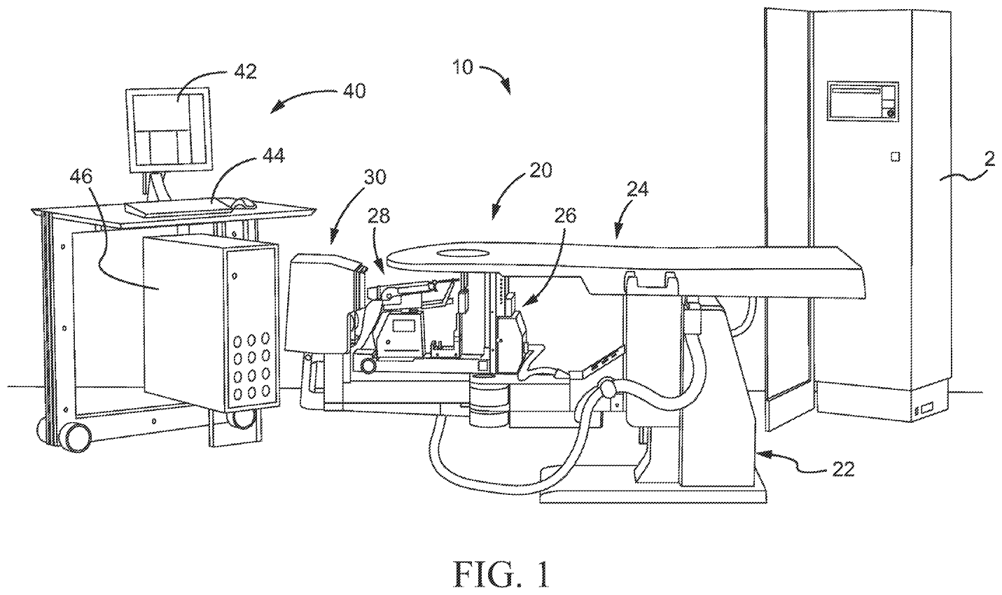

FIG. 1 shows an exemplary stereotactic, also known as “X-Ray” biopsy suite (10). Suite (10) comprises a support assembly (20), a control module (40), and an X-ray generator (2). Support assembly (20) is connected to control module (40) and X-ray generator (2) via cables (not shown). Generally, and as will be described in greater detail below, support assembly (20) is operable to support a patient and immobilize the patient's breast to fix the breast relative to a fixed three-dimensional Cartesian coordinate system. With the patient's breast immobilized, support assembly (20) may be used to provide a plurality of radiographs using X-rays generated by X-ray generator (2). Control module (40) may then be used by an operator to analyze the radiographs. Specific locations of interest within the patient's breast may then be identified and their specific Cartesian coordinates stored using control module (40). Support assembly (20) may then be used to assist an operator with targeting the locations of interest with an attached biopsy device to extract tissue samples.

Some merely exemplary biopsy devices that may be used with suite (10) are disclosed in U.S. Pat. No. 7,854,707, entitled “Tissue Sample Revolver Drum Biopsy Device,” issued Dec. 21, 2010; U.S. Pat. No. 8,083,687, entitled “Tissue Biopsy Device with Rotatably Liked Thumbwheel and Tissue Sample Holder,” issued Dec. 27, 2011; and U.S. Pat. No. 8,241,226, entitled “Biopsy Device with Rotatable Tissue Sample Holder,” issued Aug. 14, 2012, the disclosure of which is incorporated by reference herein. Alternatively, suite (10) may be used with any other biopsy devices, including but not limited to any of the biopsy devices disclosed in any of the various references that are incorporated by reference herein.

Control module (40) comprises a display screen (42), a user input apparatus (44), and a data processing and storage unit (46). By way of example only, display screen (42) may comprise a conventional computer monitor; user input apparatus (44) may comprise a conventional keyboard and mouse; and data processing and storage unit (46) may comprise a conventional computer that is modified to include software operable to execute the processes described herein. As will be described in greater detail below, control module (40) is configured to obtain and store radiographic images, execute various image processing algorithms, and display radiographic images based on user input for analysis. Although control module (40) is shown as having a particular configuration, it should be understood that control module may be configured in any suitable way as will be apparent to those of ordinary skill in the art in view of the teachings herein.

Support assembly (20) of the present example includes a base assembly (22) supporting a patient table (24), a breast compression assembly (26), a biopsy device guide assembly (28), and an x-ray tube assembly (30). Generally, base assembly (22) is adjustable to position table (24), breast compression assembly (26), biopsy device guide assembly (28), and x-ray tube assembly (30) relative to each other. For instance, in some examples, a patient is positioned in a prone position on table (24). Table (24) is configured such that one or more of a patient's breasts may extend downwardly though table (24) such that fixation of one or more breasts can be achieved using breast compression assembly (26). Once secured therein, the patient remains substantially stationary while biopsy device guide assembly (28) and x-ray tube assembly (30) are positioned relative to a patient.

In some examples, a stereoscopic imaging procedure is performed by pivoting x-ray tube assembly (30) into different stereotactic positions. Generally, this can involve pivoting x-ray tube assembly (30) into a first position at +15° (or some other angle) and then a second position at −15° (or some other angle) relative to its initial position. Radiographs can be taken at each position and then control module (40) may triangulate specific regions of interest using triangulation. Regions of interest may then be targeted by a breast biopsy device under the guidance of biopsy device guide assembly (28). It should be understood that specific angular values provided herein are merely illustrative and in other examples numerous other angular values may be used. Moreover, while the various devices, configurations, features and methods are described herein in connection with a stereotactic biopsy suite (10), such devices, configurations, features and methods may be readily used in connection with other alternative biopsy suites. By way of example only, suitable biopsy suites may include ultrasound suites, MRI suites, and any other suitable kind of biopsy suite as will be apparent to those of ordinary skill in the art in view of the teachings herein.

II. Exemplary Biopsy Process

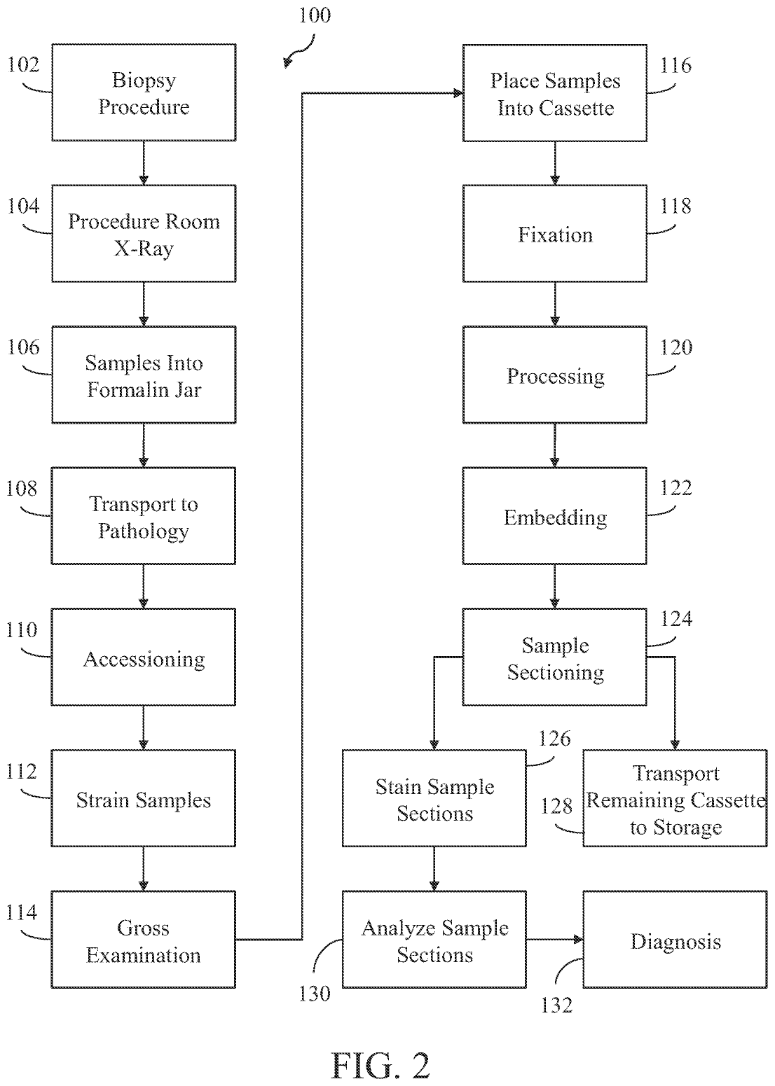

Tissue samples may be subjected to various processing and or analysis steps after the tissue samples are collected a biopsy device or other suitable devices. During such steps, collected tissue may be transported, tracked, and stored multiple times at various stages. For example, FIG. 2 shows a general workflow (100) that may be associated with a biopsy process. It should be understood that the workflow (100) shown in FIG. 2 and the description herein is only exemplary and that various alternative procedural steps may be used in addition and/or in the alternative to the steps shown in FIG. 2. For instance, in some examples, one or more steps may be performed in accordance with one or more of the teachings of U.S. Ser. No. 15/638,843, entitled “Integrated Workflow for Processing Tissue Samples from Breast Biopsy Procedures,” filed on Jun. 30, 2017, the disclosure of which is incorporated by reference herein.

In the workflow (100) shown in FIG. 2, tissue samples are collected during a biopsy procedure (block 102). During the biopsy procedure in (block 102), a biopsy device may be used to collect a plurality of tissue samples. After collection, samples may be subjected to a procedure room x-ray (block 104). During a procedure room x-ray, an operator uses x-ray imaging in the procedure room to perform preliminary analysis on the collected tissue samples. During this stage, the collected tissue samples are primarily analyzed using x-ray imaging to determine if any one or more of the collected tissue samples include calcifications or other suspicious features identifiable via x-ray. After this preliminary analysis, more tissue samples can be acquired, if an operator is not satisfied with the preliminary analysis. Alternatively, an operator may be satisfied with the originally collected tissue samples and move to the next step in the procedure.

After an operator is satisfied with preliminary procedure room x-ray analysis, the operator may insert the sample into a formalin jar (block 106) or other storage container. A formalin jar may be filled with formalin or other fluids to preserve the collected tissue samples for storage and/or transport. The formalin jar or other tissue storage container may then be transported to a pathology laboratory for further analysis (block 108).

After being received by the pathology laboratory, the samples can be subjected to accessioning (block 110). Accessioning (110) may refer to the process of documenting the chain of custody of the collected tissue samples. It should be understood that this may include a variety of steps. For instance, in some examples, a sample container can include a label that can be used to store, present, display, or otherwise provide patient information. This label can be printed during or after the biopsy procedure described above (block 102). The label can then be adhered to a formalin jar or other container prior to transport to pathology (block 108). Once received by pathology, an operator can record, scan, or otherwise collect information from the label to track the chain of custody of the collected tissue samples.

Once accessioning is complete, the collected tissue samples are strained from the fluid contained within a formalin jar or otherwise removed from a container (block 112). The collected tissue samples then undergo gross examination by an operator (block 114). Gross examination can include visual inspection of the collected tissue samples, palpitation of the collected tissue samples, and/or manipulating the collected tissue samples into a desired position. Preliminary observations can then be documented in a written record by an operator. Such written or electronic records can then be associated with the label or other identifier described above (block 110).

After gross examination or during gross examination, the collected tissue samples may be placed into other storage containers or cassettes for further analysis (block 116). For instance, in some cases each collected tissue sample may be placed into a tissue cassette that is adapted to aid in tracking the sample, transporting the sample, or analyzing the sample with an automated sample testing device. To promote tracking of the collected tissue samples, the tissue processing cassette can be labeled at this stage by either direct printing or adhering a self-adhering label to the container or cassette. This label can include certain patient information corresponding to the label described above with respect to accessioning (block 110).

Once the collected tissue samples are disposed within a tissue processing cassette or other container, the collected tissue samples may be further prepared and subjected to fixation (block 118). The term fixation used herein refers to the process of using a fixative to preserve specimen integrity and to maintain the shape of cells. Generally, this process involves submerging the collected tissue samples within a fixative. One common fixative is 10% neutral buffered formalin, although other fixatives can be used. The collected tissue samples can be maintained within the fixative for a predetermined period of time. Suitable periods of time can vary according to a variety of factors. However, under many circumstances, a suitable period of time can be approximately 6 hours. This period is generally sufficient to provide stabilization of the proteins in the collected tissue samples to substantially prevent degeneration of the collected tissue samples.

After fixation is complete, the collected tissue samples are subjected to various chemical solutions (block 120). During this process, multiple tissue processing cassettes may be loaded into a basket for bulk processing. Various chemicals are then applied, which may enter each tissue processing cassette or other container. Various chemicals may be used during this process such as alcohols of various concertation levels. For instance, when alcohol is used, moisture is removed from each collected tissue sample rendering each collected tissue sample hard in texture and generally dehydrated.

Once processing is complete, the collected tissue samples are subjected to an embedding process (block 122). During the embedding process, the collected tissue samples are surrounded by a histological wax. In one merely exemplary embedding process, the tissue samples are removed from the tissue processing cassette and placed into a metal tray or container. Prior to placement of the tissue samples within the metal tray, the metal tray can be partially filled with an initial amount of molten wax. Once the samples are placed in the metal tray, the metal tray is then filled with molten wax. The tissue processing cassette is then placed on the top of the metal tray with the underside of the cassette facing the tissue samples. Additional molten wax is then added through the cassette to bond with wax in the metal tray. During this process, the metal tray can be placed on a cold plate or other cold surface to provide relatively quick solidification of the wax. Once solidification is complete, the collected tissue samples and cassette can be removed from the metal tray. It should be understood that once the tissue samples are prepared in this manner, the tissue samples are generally preserved for indefinite storage at room temperature.

After the embedding process is complete, thin slices of each collected tissue sample are acquired (block 124). Sample sectioning may be performed using a microtome machine. Such a machine uses precision blades to slice thin samples longitudinally from each collected tissue sample. The thin sections are then placed on slides for viewing under suitable visualization means such as optical microscopes.

Once the tissue sample sections are placed on a slide, the sections are subjected to staining (block 126). The portion of the collected tissue samples that remain in the tissue processing cassette are transported to storage (block 128). During the staining process, various chemical compounds are applied to the tissue sample sections. Each chemical compound may be configured to react to different tissue cells. For instance, some compounds may be configured to specifically react with cancer cells, thereby staining cancer cells with a distinctive color relative to other cells. Although not represented in FIG. 2, it should be understood that in some examples the staining process can include multiple stages of staining. For instance, in some examples staining can include primary staining followed by advanced staining.

Once staining is complete, the stained sample sections are analyzed by an operator using a microscope or other visualization means (block 130). Based on this analysis a diagnosis may be generated (block 132).

III. Exemplary Risk Assessment System

As can be seen from the steps of FIG. 2, there are numerous steps of a biopsy procedure, some requiring transport of a sample between two locations or coordination of activity between two or more persons. In order to minimize the potential for error or miscommunication during the procedure, and maximize accuracy of a diagnosis across numerous and complex steps, it may be helpful to audit or assess the practices and procedures in use at a facility. Self-assessment and third-party interviews of personnel may not provide a clear picture or actual practices within a facility, and so it may be advantageous and desirable to provide a guided assessment that a third-party auditor may perform based upon objective, observable factors. However, providing such an assessment is not a trivial task, as the steps of FIG. 2 include as many as 730 different factors that may contribute to error, as will be explained in further detail below. Providing an assessment with more than seven hundred questions will be undesirable for most implementations, and so a guided assessment may advantageously provide a subset of questions that each address multiple points of risk.

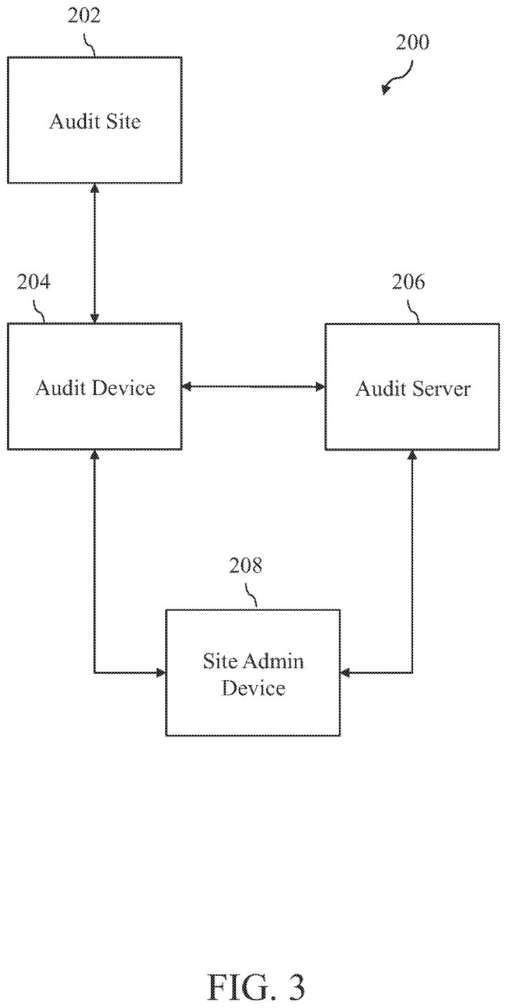

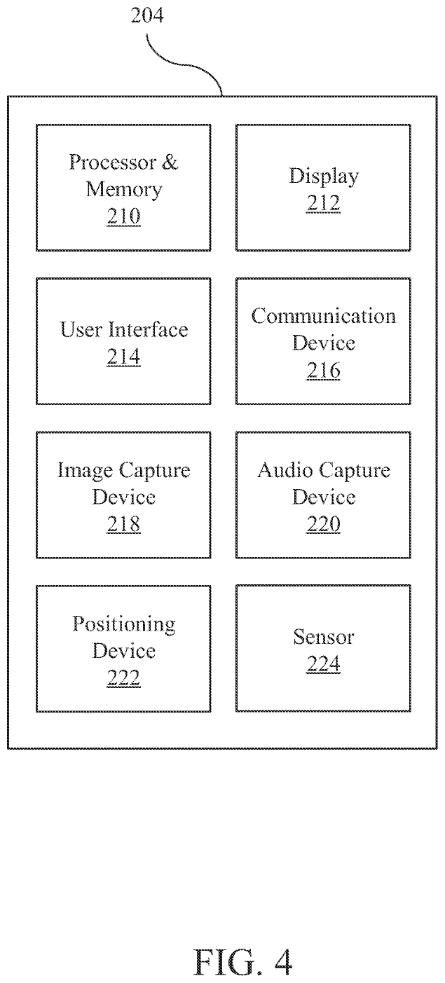

Turning now to FIG. 3, that figure shows a diagram of an exemplary assessment system (200) that may be used to provide such an assessment. The system (200) includes an audit device (204) operable by an auditor to complete an assessment for an audit site (202). The audit device (204) may be a computer such as a laptop computer, a handheld device such as a tablet or smartphone, or a proprietary device or other computing device having components such as a processor, memory, display, user input, communication device, and others capable of sending and receiving data with other devices and networks, analyzing information, manipulating information, and operating integrated or attached devices. As an example, FIG. 4 shows an exemplary audit device such as the audit device (204). The audit device (204) includes a processor and memory (210) that may be configured to store and execute software instructions in order to analyze and manipulate data, and control the function of other components and devices of the audit device (204). The audit device (204) also includes a display (212), operable to provide information to a user, a user interface (214) such as a software keyboard input operable to receive information from a user, and a communication device (216) operable to send and receive information across networks and with other devices.

The audit device (204) may also include an image capture device (218), which may be a camera or other optical capture device, which may be configured to capture photographic images, scan barcodes or other optical identifiers, or similar features. Image capture may be useful during an assessment in order to capture photographic images at the audit site (202) of rooms, tools, processes, or other characteristics related to the assessment. For example, one assessment module may concern the manner or area in which sample containers are stored. In such an example, the image capture device (218) may be used to capture an image of the sample container as observed by the auditor, which may be used to review or support the results of an assessment (e.g., an observable risk indicator image may be used to evaluate an observable risk indicator). The audit device (204) may also include an audio capture device (220), which may be used to capture an auditor's spoken notes or comments related to a particular assessment module, or may, where appropriate, include responses from site personnel related to an assessment module.

The audit device (204) may also include a positioning device (222), which may be operable to provide a location where the audit device (204) is located relative to the audit site (202). This may include a global positioning device, or may also include a feature of the communication device (216) which may enable wireless triangulation or beacon based location determination within the audit site (202). Position of the audit device (204) may be useful to capture and associate with captured images or audio, to determine the auditor's location within the audit site (202) and provide an appropriate assessment module (e.g., after determining that the audit device (204) is located in the biopsy suite (10), provided guided assessment modules relating to the biopsy suite (10)). The audit device (204) may also include other sensors (224) of varying function, which may include accelerometers, altimeters, and other devices allowing for determination of movement, including step counting, stair climbing, and other activities. Functionality of the sensor (224) that can determine step distance, stair climbing, or other distance determinations between points may be useful to capture such information during assessment without using a tape measure or other tool. For example, one assessment module may require an auditor to determine the distance between a location where empty sample storage containers are placed, and where filled sample storage containers are stored until transport. Variations and additional features present in the audit device (204) exist and will be apparent to one of ordinary skill in the art in light of this disclosure.

The audit site (202) may be one or more facilities, rooms, buildings, or other locations such as hospitals, laboratories, or other areas where one or more of the steps of FIG. 2 may be performed. The system (200) may also include an audit server (206), which may be one or more servers, including physical, virtual, and remote or hosted servers, and databases, which may be configured to store and communicate data relating to assessments of audit sites. In varying implementations, the audit server (206) may be configured to receive audit results from the audit device (204), provide guided assessment software, software updates, or software configurations, and other similar features. The system (200) may also include a site admin device (208), which may be a similar device as the audit device (204), but may be associate with a personnel or administrator of the audit site (202). The audit device (204) may be configured to receive assessment results or other information from the audit device (204) or the audit server (206).

IV. Methods for Risk Assessment

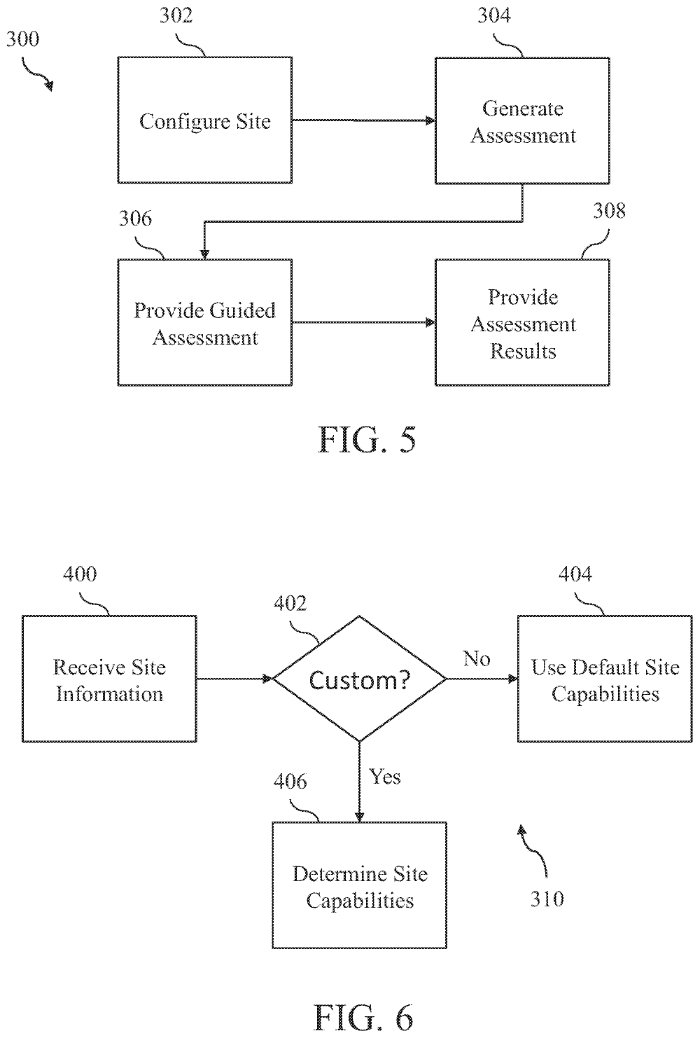

Turning now to FIG. 5, that figure shows an exemplary set of high level steps (300) that may be performed by one or more components of the system (200), such as the audit device (204), in order to complete a guided assessment. A site such as the audit site (202) may be configured (302) with the system (200) in order to provide a personalized guided assessment. For example, some audit sites may not include each capability shown in FIG. 2, and so a guided assessment including modules based upon non-existent capabilities may confuse auditors or produce erroneous results. The system (200) may then generate (304) the guided assessment by selecting one or more assessment modules to be included in the assessment. The system (200) may then provide (306) the guided assessment to the auditor, in the form of a software interface, software application, or other software experience provided via a device such as the auditor device (204). After the assessment is completed, the system (200) may then provide (308) the assessment results, which may include providing various information via the audit device (204), providing information to the audit server (206), the site admin device (208), or other devices.

While FIG. 5 shows high level steps (300) for completing a risk assessment, FIGS. 6-9 show more details versions of exemplary steps that may be performed during one or more of the steps of FIG. 5. Turning now to FIG. 6, that figure shows an exemplary set of steps (310) that may be performed to configure the system (200) for assessment of a site such as the audit site (202). The system (200) may receive (400) site information associated with the audit site (202), which may include its name, description, location, contact information, or a unique identifier such as a site number, which may be used to determine one or more capabilities available at the site (e.g., some site capabilities may end at transport to pathology (108), while other site capabilities may begin at accessioning (110)). Site capabilities may be received (400) directly as manual selections from a list of capabilities, or may be determined based upon information identifying the site and accessibility to a database of site capabilities (e.g., a site may be uniquely identified by a received (400) street address, and a database may contain data indicating the capabilities at that site).

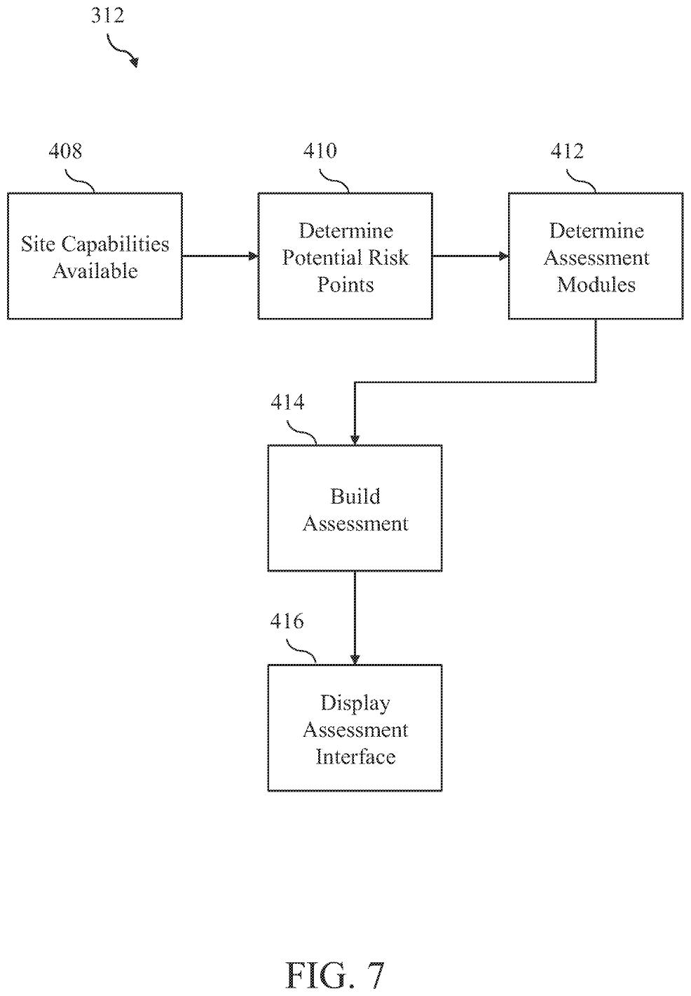

A user may then choose whether a custom (402) assessment is needed for the audit site (202). Where a custom assessment is not needed (402), such as where the audit site has a full set of assessable capabilities, a set of default site capabilities may be used (404). Where a custom assessment is needed (402), the sites actual capabilities (406) may be determined based upon the received (400) information. Turning now to FIG. 7, that figure shows an exemplary set of steps (312) that may be performed to build a guided assessment for a site such as the site (202). Once the site capabilities are available (408), one or more potential risk points associated with those site capabilities may be determined (410). Potential risk points may be determined (410) using a dataset of identified risks associated with site capabilities, which may include, for example, selecting a set of records from a database based upon the one or more site capabilities. Some discussion of risk points and their associated may be useful, in the context of FIG. 7 and determining (410) the potential risk points. As an example, Table 1 shows a set of exemplary capabilities, as well as a number of identified risk points associated with each capability.

TABLE 1
Exemplary risk points associated with Site Capabilities

| Capability | Risk Points |
|---|---|
| Radiology | 134 |
| Transfer to Pathology | 69 |
| Grossing | 92 |
| Processing and Fixation | 56 |
| Embedding | 34 |
| Microtomy | 68 |
| H&E Staining | 53 |
| Coverslipping | 53 |
| Slide Prep | 29 |
| Immunohistochemistry (IHC) | 146 |

With reference to Table 1, where a site has capabilities for performing radiology tasks (e.g., performing a biopsy (102), performing an x-ray (104), and storing samples (106)), there are 134 identified risk points. Additional information for these risk points may also be accessible to the system (202), and for example, may be stored in a database (e.g., a risk database) and selectable based upon their association with a capability. As an example, Table 2 provides a set of exemplary risk points associated with a radiology capability. A step column describes a particular step within a radiology capability associated with the risk factor, while risk describes the potential risk and result (e.g., if patient records are mislabeled, it may lead to an incorrect diagnosis), severity or severity score describes the magnitude of the risk when it occurs on a numerical scale, occurrence or occurrence score describes the likelihood of the risk occurring on a numerical scale, and indicators describe one or more observable factors associated with the risk (e.g., where an auditor observes batch patient processing, stacks of labels, or a printer in a separate room from the biopsy suite (10), that risk is implicated). The numerical scale on which severity and occurrence are represented may vary, and may include true numeric scales (e.g., 1-10, 1-100) or limited numeric scales (e.g., where the scale is expressed as limited number of options, and each option is associated with a numeric value, such as where a low severity is equal 1, a moderate severity is equal to 3, and a high severity is equal to 9).

TABLE 2
Exemplary risk points associated with tadiology capability

| Step | Risk | Sev. | Occ. | Indicators |
|---|---|---|---|---|
| Room | Incorrect label and | 9 | 3 | Batch patient |
| set up | incorrect diagnosis | | | processing |
| | | | | Label stacks |
| | | | | Separate printer |
| Room | Missing label and | 3 | 9 | Batch patient |
| set up | delay in diagnosis | | | processing |
| | | | | Label stacks |
| | | | | Separate printer |
| Room | Incorrect pre-operative | 1 | 3 | Batch files in rooms |
| set up | images and delay in | | | Unlabeled images |
| | diagnosis | | | |
| Room | Incorrect specimen | 3 | 1 | Unidentified |
| set up | collection and | | | collections jars |
| | rebiopsy | | | Unidentified |
| | | | | collection fluid |
| | | | | Collection in |
| | | | | petri dish |
| Room | Incorrect specimen | 9 | 1 | Unidentified |
| set up | collection and | | | collections jars |
| | incorrect diagnosis | | | Unidentified |
| | | | | collection fluid |
| | | | | Collection in |
| | | | | petri dish |
| Set up | Device and software | 9 | 1 | Manual software entry |
| biopsy | mismatch and | | | Default software |
| device | incorrect diagnosis | | | configuration |
| Set up | Device and software | 3 | 1 | Manual software entry |
| biopsy | mismatch and delay in | | | Default software |
| device | diagnosis | | | configuration |

Each risk point in a risk point dataset or table, such as that shown in Table 2, may also be associated with other data related to the guided assessment. For example, Table 3 shows a similar dataset as that of Table. 2, additionally associated with a post control occurrence for each identified risk point. The post control occurrence indicates the likelihood of that risk occurring after implementing procedures or controls related to the indicators for that risk point. For example, where a label printer is observed as being in a separate room from the biopsy suite (10), there is a risk of patient data or samples not being labeled leading to a significant delay in diagnosis, with an occurrence of 9 (e.g., extremely likely to occur). After implementing controls to mitigate the indicator, such as moving the label printer into the biopsy suite (10), the post control occurrence indicates that the likelihood of occurrence is reduced to 1 (e.g., extremely unlikely to occur).

TABLE 3
Exemplary risk points post-control

| | | | | Post |
|---|---|---|---|---|
| | | | | Control |
| Step | Risk | Sev. | Occ. | Occ. |
| Room | Incorrect label and | 9 | 3 | 1 |
| set up | incorrect diagnosis | | | |
| Room | Missing label and | 3 | 9 | 1 |
| set up | delay in diagnosis | | | |
| Room | Incorrect pre-operative | 1 | 3 | 1 |
| set up | images and delay in | | | |
| | is diagnosis | | | |
| Room | Incorrect specimen | 3 | 3 | 1 |
| set up | collection and | | | |
| | rebiopsy | | | |
| Room | Incorrect specimen | 9 | 3 | 1 |
| set up | collection and | | | |
| | incorrect diagnosis | | | |
| Set up | Device and software | 9 | 3 | 1 |
| biopsy | mismatch and | | | |
| device | incorrect diagnosis | | | |
| Set up | Device and software | 3 | 3 | 1 |
| biopsy | mismatch and delay in | | | |
| device | diagnosis | | | |

With full datasets and risk points for each capability, as shown in Tables 1-3, it can also be seen that the system (10) may determine in real-time or store other data associated with risk points. For example, Table 4 shows a total pre-control risk, and a total post-control risk (e.g., individual post-control risk scores, occurrence scores, severity scores, aggregate post-control risk scores, occurrence scores, severity scores) associated with each capability. These figures may be determined by, for example, multiplying the severity of each risk point with the occurrence of each risk point (e.g., severity of nine multiplied by occurrence of three equals a total risk of twenty-seven), and summing the total risk by each capability. Total pre-control risk may indicate the total risk for a site that where indicators for each risk point are observed. Post-control risk may be determined by, for example, multiplying the severity of each risk point with the post-control occurrence of each risk point, and summing the total risk by each capability. Post-control risk may indicate the total risk for a site where each indicator has been controlled or accounted for. Totaled or aggregated risk scores may also be referred to herein as “total risk scores,” and may refer to different aggregate values based upon the input values used to arrive at the total risk score. As an example, risk scores may be aggregated to provide total risk scores for particular steps, particular procedures, particular instruments, particular procedure sites, or other characteristics. Such information, as shown in Table 4, may be useful to provide results of an assessment, as will be described in more detail below.

TABLE 4
Exemplary pre-control and post-control risk by capability

| | | Pre- | Post- |
|---|---|---|---|
| | Risk | Control | Control |
| Capability | Points | Risk | Risk |
| Radiology | 134 | 1606 | 1092 |
| Transfer to Pathology | 69 | 1523 | 1127 |
| Grossing | 92 | 1518 | 1123 |
| Processing and Fixation | 56 | 1061 | 923 |
| Embedding | 34 | 1199 | 935 |
| Microtomy | 68 | 1243 | 907 |
| H&E Staining | 53 | 1215 | 960 |
| Coverslipping | 53 | 1254 | 878 |
| Slide Prep | 29 | 1650 | 990 |
| IHC | 146 | 1672 | 1053 |

Returning to FIG. 7, after determining (410) the potential risk points, one or more associated assessment modules may be determined (412). Assessment modules may include one or more guided assessment questions for an auditor to evaluate, observe, and provide responses for, and may include, for example, video, text, or image instructions for identifying a capability or step (e.g., instructions for locating the biopsy suite (10), instructions for locating the biopsy device guide assembly (28), instructions for locating a label printer), for evaluating a capability or step for its impact on an indicator such as that shown in Table 2 (e.g., determine whether a label printer is located in the biopsy suite (10), determine the size or other characteristics of a biopsy device in use), and for providing a response for a question of the module (e.g., provide a yes or no response indicating whether the printer is in the biopsy suite (10), provide a yes or no response indicating whether a biopsy device software is manually configured.

Assessment modules may be determined (412) based upon their association with a particular capability or step. For example, where the configured site capabilities indicate that a guided assessment should include a radiology module, any assessment modules associated with aiding an auditor in evaluating indicators for radiology capability may be selected. In some implementations, modules may also be selected and included based upon association with a step rather than a capability (e.g., selected by a step from Table 2 rather than selecting based upon capability), which may allow for additional precision in selecting modules for particulars site configurations. Once the associated assessment modules have been determined (412), the guided assessment may be built (414) displayed (416) as an assessment interface. Building (414) the guided assessment may include packaging the determined (412) modules into a software interface that is configured to be installed on the audit device (204), or may include providing updated configurations or data files to the audit device (204) which is already configured with a software application or interface capable of displaying a guided assessment using the provided information.

As another example, in some implementations an assessment may be built (414) by packing the associated modules and interface into a software application that may be distributed directly to, or through software marketplaces for, devices running iOS, Android, Windows, or other mobile operating systems. In other implementations, such devices may be configured with an assessment loader application, and built (414) assessments may be provided to such devices as a packaged or encoded dataset that may be opened by the assessment loader application.

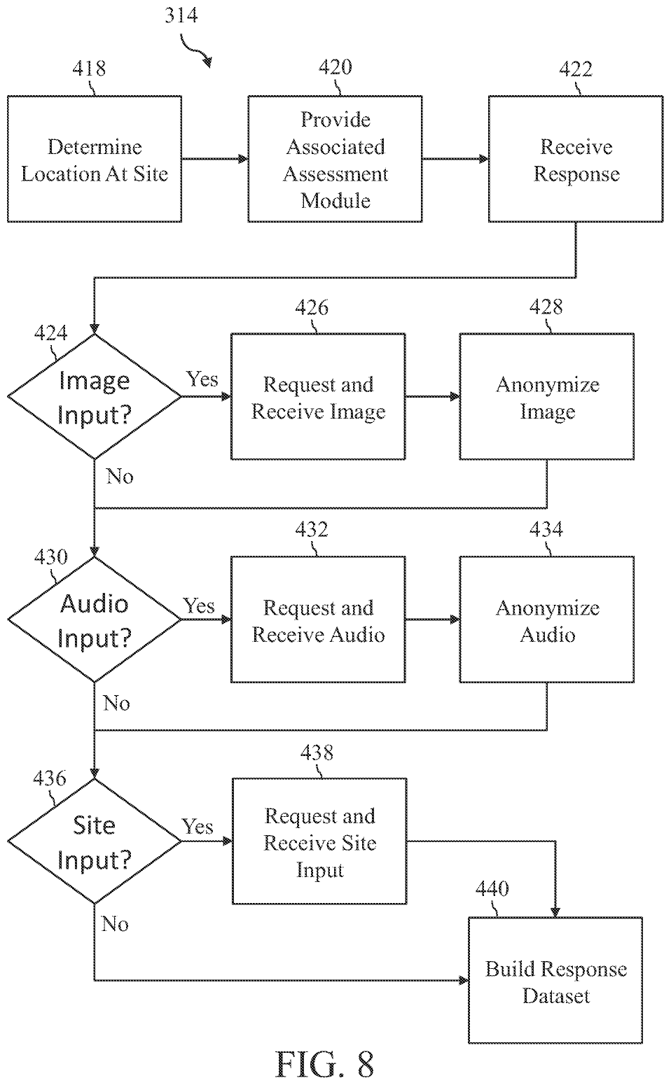

In any case, the assessment interface (416) may then be displayed via the audit device (204), which may provide the auditor instructions for performing the audit, controls and instructions for navigating through assessment modules, providing responses to assessment modules, and other steps that may be required to perform the guided assessment, as has been described. Using the provided interface, an auditor may then complete the guided assessment by observing and providing responses to the one or more assessment modules. For example, FIG. 8 shows an exemplary set of steps (314) that may be performed to provide and complete assessment modules of a guided assessment.

In some implementations, a guided assessment may present assessment modules sequentially, following an order similar to that shown in FIG. 2 (e.g., a first set of assessment modules may be for radiology, a second set of assessment modules may be for pathology), or may allow an auditor to manually select a desired set of assessment modules In other implementations, the assessment software running on the audit device (204) may determine (418) the current location of the audit device (204) based upon various factors, and may provide (420) an associated assessment module based upon the determined (418) location.

For example, in some implementations, the audit device (204) may use a positioning device (222) to determine a present location within the audit site (202). Such information may be compared to a stored site map or lookup table associated with the audit site (202) in order to determine which assessment modules are associated with nearby capabilities or steps, and which should be provided (420). In some implementations, the image capture devices (218) may be used to scan a barcode or other optical identifier associated with a location or step, such as where the biopsy suite (10) or a particular device or area of the biopsy suite (10) is marked with an optical identifier (e.g., a biopsy device may include a barcode which may be scanned and identified as being associated with a biopsy device, causing any assessment module associated with the biopsy device, capabilities, or steps to be provided via the audit device (204)). In implementations where a user may manually or sequentially access assessment modules, the audit device (204) may provide (420) the requested or subsequent assessment module, as appropriate.

After providing (420) an assessment module, the audit device (204) may receive (422) a response, which may include text and other information. This may include yes or no responses (e.g., to a question such as “Is the printer located in the biopsy suite?”, or “Are any of the following observed: Printer located separately from biopsy suite. Stacked labels. Batch processing of patients or samples.”), selections of pre-defined options (e.g., “Observe and select one of the following: (A) Printer is not located in biopsy suite, (B) Labels are stacked, (C) Batch processing of samples, (D) None of the above”), menu selections, radio button selections, or other similar inputs that may be provided in response to the guided assessment prompts.

Some assessment modules may also require or request one or more of an image input (424), an audio input (430), or a site input (436). Where an image input is required (424), the guided assessment may provide a camera interface for capturing images using the image capture device (218), while also displaying text describing the desired image. For example, where the response received indicates that a label printer is not located in the biopsy suite (10), the requested image input (424) may be an image of the printer wherever it is located. The guided assessment may then receive (426) the requested image after the user captures it, and may also take one or more steps to anonymize (428) the image to preserve the confidentiality of patients, personnel, or other aspects of the audit site (202). Anonymization (428) may include both manual and automated actions. For example, manual anonymization (428) may be achieved by displaying the received (426) image via the audit device (204) with an interface that an auditor may interact with to crop, blur, erase, or mark over any of the image contents that are not necessary for the guided assessment (e.g., a patient's face, name, or other information, personnel faces or name tags, information identifying a particular audit site or hospital). Automatic anonymization (428) may include image recognition to identify faces, limbs, text, and other aspects of an image so that they may be automatically blurred, remove, or otherwise obfuscated, or applying image wide filters or conversions in order to reduce detail in the image (e.g., converting to dot patterns, line edges, image wide blurring).

Where an assessment module may request audio input (430), audio data may be captured via the audio capture device (220). Received (432) audio may include an auditor's spoken voice, an audible alert provided by a piece of equipment that is ignored, or responses to a brief interview with personnel (e.g., where some clarification to an observable circumstance may be helpful in later analysis of the assessment). As with captured images, captured audio may be anonymized (434) in order to provide confidentiality, which may prompt more truthful responses to interviews, for example.

Where an assessment module requires some other site input (436), another sensor or device of the audit device (204) may be used to provide such information. This may include, for example, using an accelerometer or motion sensor configured to function as a step counter (e.g., a step counting device) to determine the distance between two points. Such information may be received (438) and associated with responses for some assessment modules. For example, where a particular assessment module determines whether tissue samples are appropriately transported from radiology to pathology, it may be useful to determine the number of steps taken between the biopsy suite (10) (e.g., a start location) and a sample drop off point for pathology (e.g., a destination location), as a lengthy path may be an indicator for certain risk points. As requested and required input is received, a response dataset may be built (440), which may include Boolean and textual responses and descriptions to assessment module queries, images, audio, and other information as described above.

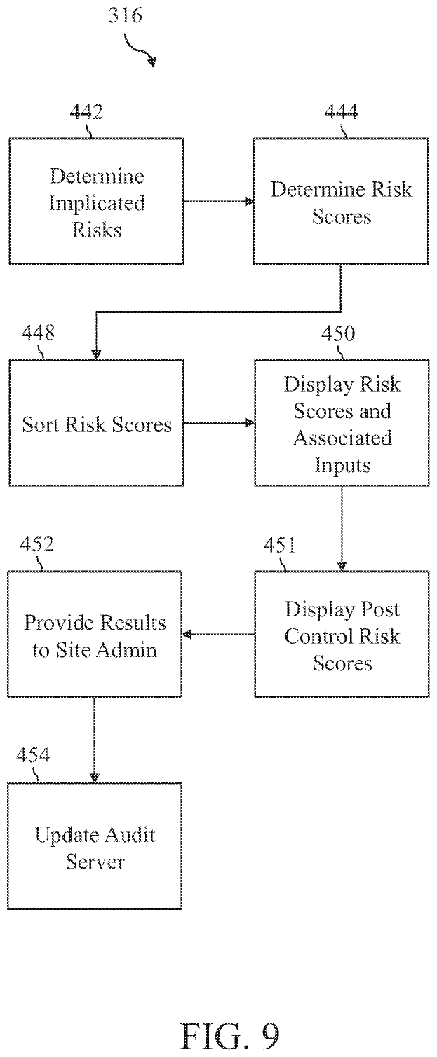

With a response dataset available, the system (200) may then determine and assess an overall risk associated with the audit site (202) based upon the auditor's observations and responses provided during the guided assessment. For example, FIG. 9 shows a set of steps (316) that may be performed to produce assessment results. The response dataset may be analyzed to determine (442) the risk points that are implicated by the contents of the response dataset. Where information provided in response to an assessment module shows that one or more indicators associated with a risk point, such as those shown in Table 2, the system (10) may determine that the risk point has not been controlled for, and has a pre-control occurrence rate (i.e., as opposed to a lower post-control occurrence rate). As an example with reference to Table 2, where responses to an assessment module indicate that a label printer is located in a different room than the biopsy suite (10), two risk points may be determined (442) to be implicated: incorrect label resulting in incorrect diagnosis, and missing label resulting in delay in diagnosis.

With one or more risk points implicated, the system (10) may then determine (444) risk scores associated with each risk point. A risk score may indicate an overall level of risk associated with that particular risk point, and may be expressed as a product of the severity of a risk (e.g., on a scale of 1, 3, or 9 as shown in Table 2) and the occurrence rate of the risk (e.g., on a scale of 1, 3, or 9 as shown in Table 2, with a resulting range of risk of 1, 3, 9, 27, or 81). With the above example, where the risks of incorrect label resulting in incorrect diagnosis, and missing label resulting in delay in diagnosis are implicated, the risk for each may be determined (444) as twenty-seven (i.e., incorrect diagnosis is a severity of 9 and occurrence of 3, while delay in diagnosis is a severity of 3 and an occurrence of 9).

After determining (444) the implicated risk scores, the system (10) may sort (448) and display (450) the risk scores in various ways. In some implementations, the risk scores and risk points may be sorted (448) from highest risk to lowest risk, and displayed (450) via the audit device (204) or another device in order to provide guidance on the highest risk indicators identified at the audit site (202). For example, with reference to Table 2, if each risk point is implicated for the audit site (202), each may be displayed, with incorrect label resulting in incorrect diagnosis, and missing label resulting in delay in diagnosis being sorted (448) to the top of the list, since each has a total risk of twenty-seven, and would be equal in being the highest implicated risk points.

In some implementations, risk points may instead be sorted by capability or step, rather than individual risk point. This sort of aggregation may be useful, since some implementations of the system (10) may have nearly 750 separate risk points. Comparatively, there may be about 100 separate clinical steps, and 10 separate capabilities as shown in Table 1. For example, with reference to Table 1, each shown capability may displayed (450) along with a total implicated risk score for that capability (e.g., the sum of any implicated risks points falling within that capability, or a capability total risk), with the highest risk capability sorted to the top of the list. Such an interface may provide auditors more generalized areas of a biopsy process that may be focused on when providing controls or implementing control procedures.

The system (10) may also determine and display (451) post control risk scores for each displayed risk point, step risk, or capability risk. For example and with reference to Table 4, where the system (10) displays (450) risk scores grouped and sorted (448) by capability, a post-control risk may also be displayed (451) for each capability. Other information may also be displayed with the post-control risk, including a percentage of change from observed risk (e.g., where radiology observed risk totals to 1606, and post-control risk totals to 1092, a percentage reduction of 32% may be displayed), and a color coded risk level (e.g., a risk of 0-1000 may be a low risk and be colored within a green gradient, 1100-1700 may be a moderate risk and colored within a yellow gradient, and 1700 or higher may be a high risk and colored within a red gradient).

When displaying (450) risk scores, associated inputs may also be accessible and displayable via the audit device (204) or another device. This may include, for example, photographic images, audio output, or other information captured during various portions of the assessment. This may be useful where, for example, a site administrator may request an example or a proof of an observed risk indicator (e.g., an observable risk indicator) at the audit site (202). In such a case, the associated images may be browsed by risk point, clinical step, or capability, for example. In addition to displaying scores, the audit device (204) may also provide (452) results to one or more site administrators as an electronic transmission to the site admin device (208). Provided (452) results may be a full result set (e.g., the full text and any other associated inputs viewable by an auditor), or may be a limited or aggregated set of results or a subset of results (e.g., a brief list of pre-control risk and post-control risk grouped and sorted by capability). The audit device (204) may also update (454) the audit server (206), providing the entirety of or a subset of the result set. Such results may be useful for updating risk calculations, producing additional assessment modules, and providing historical tracking of site audits.

V. Exemplary Risk Points and Assessment Module Content

Table 5 below shows a set of queries that may be provided in an assessment module in order to determine if one or more risk points are implicated. The impact of each risk point may be assessed for the audit site (102) based upon an evaluation of each query in Table 5, which advantageously provides around 40 queries that may be used to assess a much greater number of risk points. For example, Table 6 below shows a set of risk points corresponding and implicated by the first two assessment queries for each category of Table 5 (e.g., Q1 and Q2 of Radiology, Q1 and Q2 of transfer, etc.). In table 6, column A describes the associated capability (e.g., ‘R’ for Radiology, ‘TI’ for Transfer to Pathology, ‘G’ for Grossing, ‘P’ for Processing and Fixation, ‘E’ for Embedding, ‘M’ for Microtomy, ‘Stain’ for H&E Stain or Hematoxylin and Eosin Stain, ‘CVS’ for Coverslipping, ‘Prep’ for Slide Preparation, and ‘IHC’ for Immunohistochemistry), column B describes a clinical step associated with the risk, column C describes a mode of failure associated with the risk, column D describes an effect of the mode of failure, column E describes a severity associated with that effect, column F describes a cause of the severity, column G describes a likelihood of occurrence associated with the risk, column H describes a lead indicator or risk indicator that is associated with and implicates the occurrence of the risk (e.g., when observed, the lead indicator creates a risk of magnitude equal to the severity of the risk multiplied by the likelihood of occurrence of the risk), and column I describes a post-control occurrence of the risk (e.g., a likelihood of occurrence of the risk after the lead indicator is mitigated or controlled for).

TABLE 5
Exemplary queries for assessment module by category

| Category/Capability | Assessment Query |
|---|---|
| Radiology (R): Q1 | Utilization of core needle? |
| Q2 | Using device that fires? |
| Q3 | Lack of back up device? |
| Q4 | Trained appropriately? |
| Transfer (TI): Q1 | Using pre-filled jars with leak proof seal? |
| Q2 | Large batch processing? |
| Q3 | Large volumes of paperwork? |
| Grossing (G): Q1 | Batch processing? |
| Q2 | Lack of visual cues? |
| Q3 | Lack of barcoding? |
| Q4 | Using pre-filled jars with leak proof seal? |
| Processing/Fixation (P): Q1 | Batch processing? |
| Q2 | Lack of back up device? |
| Q3 | Lack of visual cues? |
| Q4 | New staff or high turnover? |
| Q5 | Cluttered or dirty work area? |
| Embedding (E): Q1 | Batch processing? |
| Q2 | Lack of visual cues? |
| Q3 | Lack of PM logs? |
| Q4 | Manual embedding? |
| Q5 | Cluttered or dirty work area or receptacles? |
| Microtomy (M): Q1 | Batch processing? |
| Q2 | Insufficient experience or number of staff? |
| Q3 | Lack of PM logs? |
| Q4 | High turnover of staff? |
| Q5 | High volume of work with low time? |
| H&E (Stain): Q1 | Batch processing? |
| Q2 | Insufficient experience of staff? |
| Q3 | Large volume of work with limited time? |
| Q4 | Multi-platform baskets? |
| Cover slipping (CVS): Q1 | Batch processing? |
| Q2 | New staff or high turnover? |
| Q3 | Large volume of work with limited time |
| | or high volume of work with low time? |
| Q4 | Poor maintenance? |
| Slide Prep (Prep): Q1 | Stacks of paper? |
| Q2 | Manual matching? |
| Q3 | Stacks of slides? |
| Q4 | Lack of available space? |
| IHC (IHC): Q1 | Batch processing? |
| Q2 | Lack of backup device? |
| Q3 | Lack of specimen tracking? |
| Q4 | New staff or high turnover? |
| Q5 | Large volume of work with limited time |
| | or high volume of work with low time? |

TABLE 6
Exemplary Risk Points

| A | B | C | D | E | F | G | H | I |
|---|---|---|---|---|---|---|---|---|
| R | Acquire Tissue | No tissue/no | Hematoma/pain/ | 3 | Multiple insertion | 9 | Utilization of core needle, | 1 |
| | | sample acquired | damage to not | | | | firing device | |
| | | | biopsied breast | | | | | |
| | | | tissue | | | | | |
| R | Acquire Tissue | Inadequate | Incorrect Dx | 9 | Multiple insertion | 3 | Utilization of core needle, | 1 |
| | | quality tissue | | | | | firing device | |
| R | Acquire Tissue | Inadequate | Rebiopsy | 3 | Multiple insertion | 3 | Utilization of core needle, | 1 |
| | | quality tissue | | | | | firing device | |
| R | Acquire Tissue | Inadequate | Delay in diagnosis | 3 | Multiple insertion | 3 | Utilization of core needle, | 1 |
| | | quality tissue | | | | | firing device | |
| R | Acquire Tissue | Acquire | Incorrect Dx | 9 | Multiple insertion | 3 | Utilization of core needle, | 1 |
| | | incorrect tissue | (including | | | | firing device | |
| | | | underestimation) | | | | | |
| R | Acquire Tissue | Acquire | Incorrect Dx | 9 | Needle deflection/firing | 1 | Utilization of core needle, specific | 1 |
| | | incorrect tissue | (including | | | | devices | |
| | | | underestimation) | | | | | |
| R | Acquire Tissue | Acquire | Rebiopsy | 3 | Multiple insertion | 1 | Utilization of core needle, | 1 |
| | | incorrect tissue | | | | | firing device | |
| R | Acquire Tissue | Acquire | Rebiopsy | 3 | Needle deflection/firing | 1 | Use of core needle, specific | 1 |
| | | incorrect tissue | | | | | devices | |
| R | Acquire Tissue | Patient harm | Hematoma/pain/ | 3 | Multiple insertion | 3 | Utilization of core needle, | 1 |
| | | (exposed | damage to not | | | | firing device | |
| | | aperture outside | biopsied breast | | | | | |
| | | skin, excessive | tissue | | | | | |
| | | bleeding) | | | | | | |
| R | Transport tissue | Tissue damage | Incorrect Dx | 9 | Forceps/crush artifact/ | 3 | Use of core needle, forceps | 1 |
| | into consumable | | | | human causing damage to | | on counter, bulk tissue | |
| | (for ST) and/or | | | | tissue through manual | | collection (ST) | |
| | formalin (for U/S) | | | | manipulation | | | |
| R | Transport tissue | Tissue damage | Rebiopsy | 3 | Forceps/crush artifact/ | 1 | Use of core needle, forceps | 1 |
| | into consumable | | | | human causing damage to | | on counter, bulk tissue | |
| | (for ST) and/or | | | | tissue through manual | | collection (ST) | |
| | formalin (for U/S) | | | | manipulation | | | |
| R | Transport tissue | Tissue damage | Delay in diagnosis | 3 | Forceps/crush artifact/ | 1 | Use of core needle, forceps | 1 |
| | into consumable | | | | human causing damage to | | on counter, bulk tissue | |
| | (for ST) and/or | | | | tissue through manual | | collection (ST) | |
| | formalin (for U/S) | | | | manipulation | | | |
| T1 | Laboratory | Patient data/ | incorrect diagnosis | 9 | transposition of specimen and | 1 | Large batch processing, | 1 |
| | Receives into | mismatch (req | | | patient (human error) | | large inventory at | |
| | general | to jar/jar to | | | | | accessioning, large volumes | |
| | accessioning/ | specimen/etc.) | | | | | of paperwork | |
| | central processing | | | | | | | |
| T1 | Laboratory | Patient data/ | rebiopsy | 3 | transposition of specimen and | 1 | Large batch processing, | 1 |
| | Receives into | mismatch (req | | | patient (human error) | | large inventory at | |
| | general | to jar/jar to | | | | | accessioning, large volumes | |
| | accessioning/ | specimen/etc.) | | | | | of paperwork | |
| | central processing | | | | | | | |
| T1 | Laboratory | Patient data/ | delay in diagnosis | 3 | transposition of specimen and | 3 | Large batch processing, | 1 |
| | Receives into | mismatch (req | | | patient (human error) | | large inventory at | |
| | general | to jar/jar to | | | | | accessioning, large volumes | |
| | accessioning/ | specimen/etc.) | | | | | of paperwork | |
| | central processing | | | | | | | |
| T1 | Laboratory | Tissue damage | rebiopsy | 3 | didn't process/timing | 1 | Large batch processing, | 1 |
| | Receives into | | | | | | KPIs, TAT, staffing | |
| | general | | | | | | schedules vs. 24 hour, | |
| | accessioning/ | | | | | | holidays, etc. | |
| | central processing | | | | | | | |
| T1 | Laboratory | Tissue damage | incorrect diagnosis | 9 | didn't process/timing | 1 | Large batch processing, | 1 |
| | Receives into | | | | | | KPIs, TAT, staffing | |
| | general | | | | | | schedules vs. 24 hour, | |
| | accessioning/ | | | | | | holidays, etc. | |
| | central processing | | | | | | | |
| T1 | Laboratory | Tissue damage | delay in diagnosis | 3 | didn't process/timing | 9 | Large batch processing, | 1 |
| | Receives into | | | | | | KPIs, TAT, staffing | |
| | general | | | | | | schedules vs. 24 hour, | |
| | accessioning/ | | | | | | holidays, etc. | |
| | central processing | | | | | | | |
| T1 | Laboratory | Tissue damage | rebiopsy | 3 | formalin ratio/leaky jar | 1 | Not using Affirm seal, not | 1 |
| | Receives into | | | | | | using PJs, specimen jars are | |
| | general | | | | | | too small, making own | |
| | accessioning/ | | | | | | formalin onsite, | |
| | central processing | | | | | | | |
| T1 | Laboratory | Tissue damage | incorrect diagnosis | 9 | formalin ratio/leaky jar | 1 | Not using Affirm seal, not | 1 |
| | Receives into | | | | | | using PJs, specimen jars are | |
| | general | | | | | | too small, making own | |
| | accessioning/ | | | | | | formalin onsite, | |
| | central processing | | | | | | | |
| T1 | Laboratory | Tissue damage | delay in diagnosis | 3 | formalin ratio/leaky jar | 3 | Not using Affirm seal, not | 1 |
| | Receives into | | | | | | using PJs, specimen jars are | |
| | general | | | | | | too small, making own | |
| | accessioning/ | | | | | | formalin onsite, | |
| | central processing | | | | | | | |
| T1 | Transport to | Tissue damage | Incorrect | 9 | Formalin issues (leak, | 3 | Not using PJ with affirm | 1 |
| | Histology Lab | | Diagnosis | | fixation time, % to specimen, | | seal | |
| | | | | | % NBF, expired formalin) | | | |
| T1 | Transport to | Tissue damage | Delay in diagnosis | 3 | Formalin issues (leak, | 3 | Not using PJ with affirm | 1 |
| | Histology Lab | | | | fixation time, % to specimen, | | seal | |
| | | | | | % NBF, expired formalin) | | | |
| T1 | Transport to | Tissue damage | Rebiopsy | 3 | Formalin issues (leak, | 3 | Not using PJ with affirm | 1 |
| | Histology Lab | | | | fixation time, % to specimen, | | seal | |
| | | | | | % NBF, expired formalin) | | | |
| T1 | Transport to | Loss of tissue | Incorrect | 9 | Formalin issues (leak, | 1 | Not using PJ with affirm | 1 |
| | Histology Lab | | Diagnosis | | fixation time, % to specimen, | | seal | |
| | | | | | % NBF, expired formalin) | | | |
| T1 | Transport to | Loss of tissue | Rebiopsy | 3 | Formalin issues (leak, | 1 | Not using PJ with affirm | 1 |
| | Histology Lab | | | | fixation time, % to specimen, | | seal | |
| | | | | | % NBF, expired formalin) | | | |
| T1 | Histology receives | Patient data/ | incorrect diagnosis | 9 | transposition of specimen and | 1 | Large batch processing, | 1 |
| | into histology lab | mismatch (req | | | patient (human error) | | large inventory at | |
| | | to jar/jar to | | | | | accessioning, large volumes | |
| | | specimen/etc.) | | | | | of paperwork | |
| T1 | Histology receives | Patient data/ | rebiopsy | 3 | transposition of specimen and | 1 | Large batch processing, | 1 |
| | into histology lab | mismatch (req | | | patient (human error) | | large inventory at | |
| | | to jar/jar to | | | | | accessioning, large volumes | |
| | | specimen/etc.) | | | | | of paperwork | |
| T1 | Histology receives | Patient data/ | Delay in diagnosis | 3 | transposition of specimen and | 3 | Large batch processing, | 1 |
| | into histology lab | mismatch (req | | | patient (human error) | | large inventory at | |
| | | to jar/jar to | | | | | accessioning, large volumes | |
| | | specimen/etc.) | | | | | of paperwork | |
| T1 | Histology receives | Tissue damage | rebiopsy | 3 | didn't process/timing | 1 | Large batch processing, | 1 |
| | into histology lab | | | | | | KPIs, TAT, staffing | |
| | | | | | | | schedules vs. 24 hour, | |
| | | | | | | | holidays, etc. | |
| T1 | Histology receives | Tissue damage | incorrect diagnosis | 9 | didn't process/timing | 1 | Large batch processing, | 1 |
| | into histology lab | | | | | | KPIs, TAT, staffing | |
| | | | | | | | schedules vs. 24 hour, | |
| | | | | | | | holidays, etc. | |
| T1 | Histology receives | Tissue damage | Delay in diagnosis | 3 | didn't process/timing | 3 | Large batch processing, | 1 |
| | into histology lab | | | | | | KPIs, TAT, staffing | |
| | | | | | | | schedules vs. 24 hour, | |
| | | | | | | | holidays, etc. | |
| T1 | Histology receives | Tissue damage | rebiopsy | 3 | formalin ratio/leaky jar | 3 | Not using Affirm seal, not | 1 |
| | into histology lab | | | | | | using PJs, specimen jars are | |
| | | | | | | | too small, making own | |
| | | | | | | | formalin onsite. | |
| T1 | Histology receives | Tissue damage | incorrect diagnosis | 9 | formalin ratio/leaky jar | 3 | Not using Affirm seal, not | 1 |
| | into histology lab | | | | | | using PJs, specimen jars are | |
| | | | | | | | too small, making own | |
| | | | | | | | formalin onsite. | |
| T1 | Histology receives | Tissue damage | Delay in diagnosis | 3 | formalin ratio/leaky jar | 9 | Not using Affirm seal, not | 1 |
| | into histology lab | | | | | | using PJs, specimen jars are | |
| | | | | | | | too small, making own | |
| | | | | | | | formalin onsite. | |
| G | Accession into | Patient data/test | rebiopsy | 3 | Error made when entering | 1 | Manual entry, batch | 1 |
| | histology | data/physician | | | | | processing, lack of checks/ | |
| | laboratory/ | mismatch | | | | | redundancy, using non- | |
| | inputing data | | | | | | barcoded, numerical labels | |
| G | Accession into | Patient data/test | Delay in diagnosis | 3 | Error made when entering | 3 | Manual entry, batch | 1 |
| | histology | data/physician | | | | | processing, lack of checks/ | |
| | laboratory/ | mismatch | | | | | redundancy, using non- | |
| | inputing data | | | | | | barcoded, numerical labels | |
| G | Accession into | Patient data/test | incorrect diagnosis | 9 | Error made when entering | 1 | Manual entry, batch | 1 |
| | histology | data/physician | from assign | | | | processing, lack of checks/ | |
| | laboratory/ | mismatch | wrong patient) | | | | redundancy, using non- | |
| | inputing data | | | | | | barcoded, numerical labels | |
| G | Sort specimens by | Patient data/test | Delay in diagnosis | 3 | Placed specimen in ″incorrect | 3 | have piles, batch processing | 1 |
| | pathologist/tissue | data/pathologist | | | pile″ | | | |
| | type | mismatch | | | | | | |
| G | Sort specimens by | Tissue damage | rebiopsy | 3 | Placed specimen in ″incorrect | 1 | have piles, batch processing | 1 |
| | pathologist/tissue | | | | pile″ | | | |
| | type | | | | | | | |
| G | Sort specimens by | Tissue damage | Delay in diagnosis | 3 | Placed specimen in ″incorrect | 1 | have piles, batch processing | 1 |
| | pathologist/tissue | | | | pile″ | | | |
| | type | | | | | | | |
| G | Print & prepare | Patient data/test | incorrect diagnosis | 9 | Human error (transposition, | 1 | Batch processing, barcode | 1 |
| | labels, cassettes, | data/physician | | | incorrect input, etc.) | | printer not on location, | |
| | slides, etc. | mismatch | | | | | stacks, pre-printed labels, | |
| | | | | | | | hand-labeled consumables | |
| G | Print & prepare | Patient data/test | rebiopsy | 3 | Human error (transposition, | 1 | Batch processing, barcode | 1 |
| | labels, cassettes, | data/physician | | | incorrect input, etc.) | | printer not on location, | |
| | slides, etc. | mismatch | | | | | stacks, pre-printed labels, | |
| | | | | | | | hand-labeled consumables | |
| G | Print & prepare | Patient data/test | Delay in diagnosis | 3 | Human error (transposition, | 3 | Batch processing, barcode | 1 |
| | labels, cassettes, | data/physician | | | incorrect input, etc.) | | printer not on location, | |
| | slides, etc. | mismatch | | | | | stacks, pre-printed labels, | |
| | | | | | | | hand-labeled consumables | |
| G | Print & prepare | Loss/damage of | incorrect diagnosis | 9 | Wrong consumable utilized | 1 | Poor organization, lots of | 1 |
| | labels, cassettes, | tissue | | | (human error) | | consumables in one area, | |
| | slides, etc. | | | | | | lack of visual cues, lack of | |
| | | | | | | | proper labeling, | |
| | | | | | | | incompatible consumables/ | |
| | | | | | | | instrumentation | |
| G | Print & prepare | Loss/damage of | rebiopsy | 3 | Wrong consumable utilized | 1 | Poor organization, lots of | 1 |
| | labels, cassettes, | tissue | | | (human error) | | consumables in one area, | |
| | slides, etc. | | | | | | lack of visual cues, lack of | |
| | | | | | | | proper labeling, | |
| | | | | | | | incompatible consumables/ | |
| | | | | | | | instrumentation | |
| G | Print & prepare | Loss/damage of | Delay in diagnosis | 3 | Wrong consumable utilized | 3 | Poor organization, lots of | 1 |
| | labels, cassettes, | tissue | | | (human error) | | consumables in one area, | |
| | slides, etc. | | | | | | lack of visual cues, lack of | |
| | | | | | | | proper labeling, | |
| | | | | | | | incompatible consumables/ | |
| | | | | | | | instrumentation | |
| G | Match specimens | Patient/data | incorrect diagnosis | 9 | Transposition of specimen | 1 | Lack of barcoding, lack of | 1 |
| | and consumables | mismatch | | | with patient (human error) | | visual cues, lack of | |
| | (pre-grossing) | | | | | | duplicate identifiers, batch | |
| | | | | | | | processing. | |
| G | Match specimens | Patient/data | rebiopsy | 3 | Transposition of specimen | 1 | Lack of barcoding, lack of | 1 |
| | and consumables | mismatch | | | with patient (human error) | | visual cues, lack of | |
| | (pre-grossing) | | | | | | duplicate identifiers, batch | |
| | | | | | | | processing. | |
| G | Match specimens | Patient/data | Delay in diagnosis | 3 | Transposition of specimen | 3 | Lack of barcoding, lack of | 1 |
| | and consumables | mismatch | | | with patient (human error) | | visual cues, lack of | |
| | (pre-grossing) | | | | | | duplicate identifiers, batch | |
| | | | | | | | processing. | |
| G | Match specimens | Patient/data | delay in process | 1 | Misplaced or missing | 3 | Lack of barcoding, lack of | 1 |
| | and consumables | mismatch | | | consumables/specimen/etc. | | visual cues, lack of | |
| | (pre-grossing) | | | | | | duplicate identifiers, batch | |
| | | | | | | | processing. | |
| G | Grossing | Patient/data | incorrect diagnosis | 9 | Human error | 3 | Lack of barcoding, lack of | 1 |
| | | mismatch | | | | | visual cues, lack of | |
| | | | | | | | duplicate identifiers, batch | |
| | | | | | | | processing. | |
| G | Grossing | Patient/data | rebiopsy | 3 | Human error | 3 | Lack of barcoding, lack of | 1 |
| | | mismatch | | | | | visual cues, lack of | |
| | | | | | | | duplicate identifiers, batch | |
| | | | | | | | processing. | |
| G | Grossing | Patient/data | Delay in diagnosis | 3 | Human error | 3 | Lack of barcoding, lack of | 1 |
| | | mismatch | | | | | visual cues, lack of | |
| | | | | | | | duplicate identifiers, batch | |
| | | | | | | | processing. | |
| P | Processing (sort | Tissue damage | incorrect diagnosis | 9 | Human error (sort incorrectly, | 1 | Lack of specimen tracking, | 1 |
| | into container) | | | | forget, drop tissue, drop | | batch processing, messy | |
| | | | | | container, etc.) | | workstation, lack of visual | |
| | | | | | | | cues, lack of identified | |
| | | | | | | | tissue cassettes, use generic | |
| | | | | | | | protocol for all tissue | |
| P | Processing (sort | Tissue damage | rebiopsy | 3 | Human error (sort incorrectly, | 1 | Lack of specimen tracking, | 1 |
| | into container) | | | | forget, drop tissue, drop | | batch processing, messy | |
| | | | | | container, etc.) | | workstation, lack of visual | |
| | | | | | | | cues, lack of identified | |
| | | | | | | | tissue cassettes, use generic | |
| | | | | | | | protocol for all tissue | |
| P | Processing (sort | Tissue damage | Delay in diagnosis | 3 | Human error (sort incorrectly, | 1 | Lack of specimen tracking, | 1 |
| | into container) | | | | forget, drop tissue, drop | | batch processing, messy | |
| | | | | | container, etc.) | | workstation, lack of visual | |
| | | | | | | | cues, lack of identified | |
| | | | | | | | tissue cassettes, use generic | |
| | | | | | | | protocol for all tissue | |
| P | Processing (sort | cross | incorrect diagnosis | 9 | Human error (sort incorrectly, | 1 | Lack of specimen tracking, | 1 |
| | into container) | contamination/ | | | forget, drop tissue, drop | | batch processing, messy | |
| | | mixup (patient/ | | | container, etc.) | | workstation, lack of visual | |
| | | data mismatch) | | | | | cues, lack of identified | |
| | | | | | | | tissue cassettes, use generic | |
| | | | | | | | protocol for all tissue | |
| P | Processing (sort | cross | rebiopsy | 3 | Human error (sort incorrectly, | 1 | Lack of specimen tracking, | 1 |
| | into container) | contamination/ | | | forget, drop tissue, drop | | batch processing, messy | |
| | | mixup (patient/ | | | container, etc.) | | workstation, lack of visual | |
| | | data mismatch) | | | | | cues, lack of identified | |
| | | | | | | | tissue cassettes, use generic | |
| | | | | | | | protocol for all tissue | |
| P | Processing (sort | cross | Delay in diagnosis | 3 | Human error (sort incorrectly, | 1 | Lack of specimen tracking, | 1 |
| | into container) | contamination/ | | | forget, drop tissue, drop | | batch processing, messy | |
| | | mixup (patient/ | | | container, etc.) | | workstation, lack of visual | |
| | | data mismatch) | | | | | cues, lack of identified | |
| | | | | | | | tissue cassettes, use generic | |
| | | | | | | | protocol for all tissue | |
| P | Processing (sort | Loss of tissue | incorrect diagnosis | 9 | Human error (sort incorrectly, | 1 | Lack of specimen tracking, | 1 |
| | into container) | | | | forget, drop tissue, drop | | batch processing, messy | |
| | | | | | container, etc.) | | workstation, lack of visual | |
| | | data mismatch) | | | | | cues, lack of identified | |
| | | | | | | | tissue cassettes, use generic | |
| | | | | | | | protocol for all tissue | |
| P | Processing (sort | Loss of tissue | rebiopsy | 3 | Human error (sort incorrectly, | 1 | Lack of specimen tracking, | 1 |
| | into container) | | | | forget, drop tissue, drop | | batch processing, messy | |
| | | | | | container, etc.) | | workstation, lack of visual | |
| | | data mismatch) | | | | | cues, lack of identified | |
| | | | | | | | tissue cassettes, use generic | |
| | | | | | | | protocol for all tissue | |
| P | Processing (sort | Loss of tissue | Delay in diagnosis | 3 | Human error (sort incorrectly, | 1 | Lack of specimen tracking, | 1 |
| | into container) | | | | forget, drop tissue, drop | | batch processing, messy | |
| | | | | | container, etc.) | | workstation, lack of visual | |
| | | data mismatch) | | | | | cues, lack of identified | |
| | | | | | | | tissue cassettes, use generic | |
| | | | | | | | protocol for all tissue | |
| P | Processing (load | Tissue damage | incorrect diagnosis | 9 | Human error (drop cassettes | 1 | Batch processing, poor | 1 |
| | processor) | | | | inside processing container, | | practice, large volume in | |
| | | | | | not seat properly, improper | | limited time, multi-platform | |
| | | | | | positioning, incorrect | | baskets/utilization | |
| | | | | | container, overfill container) | | | |
| P | Processing (load | Tissue damage | rebiopsy | 3 | Human error (drop cassettes | 1 | Batch processing, poor | 1 |
| | processor) | | | | inside processing container, | | practice, large volume in | |
| | | | | | not seat properly, improper | | limited time, multi-platform | |
| | | | | | positioning, incorrect | | baskets/utilization | |
| | | | | | container, overfill container) | | | |
| P | Processing (load | Tissue damage | Delay in diagnosis | 3 | Human error (drop cassettes | 3 | Batch processing, poor | 1 |
| | processor) | | | | inside processing container, | | practice, large volume in | |
| | | | | | not seat properly, improper | | limited time, multi-platform | |
| | | | | | positioning, incorrect | | baskets/utilization | |
| | | | | | container, overfill container) | | | |
| P | Processing (load | Processor won't | Delay in diagnosis | 3 | Human error (overfill | 3 | Batch processing, poor | 1 |
| | processor) | initiate (as result | | | chamber, lid not positioned | | practice, large volume in | |
| | | of human error) | | | correctly, dirty chamber) | | limited time, multi-platform | |
| | | | | | | | baskets/utilization | |
| P | Processing | Tissue damage | incorrect diagnosis | 9 | Device malfunction | 1 | old, outdated equipment, | 1 |
| | (reagent check) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| P | Processing | Tissue damage | rebiopsy | 3 | Device malfunction | 1 | old, outdated equipment, | 1 |
| | (reagent check) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| P | Processing | Tissue damage | Delay in diagnosis | 3 | Device malfunction | 1 | old, outdated equipment, | 1 |
| | (reagent check) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| P | Processing | Processor won't | Delay in diagnosis | 3 | Device malfunction | 1 | old, outdated equipment, | 1 |
| | (reagent check) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| P | Processing (initiate | Tissue damage | incorrect diagnosis | 9 | Device malfunction | 1 | old, outdated equipment, | 1 |
| | protocol) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| P | Processing (initiate | Tissue damage | rebiopsy | 3 | Device malfunction | 1 | old, outdated equipment, | 1 |
| | protocol) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| P | Processing (initiate | Tissue damage | Delay in diagnosis | 3 | Device malfunction | 1 | old, outdated equipment, | 1 |
| | protocol) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| P | Processing (initiate | Processor won't | Delay in diagnosis | 3 | Device malfunction | 1 | old, outdated equipment, | 1 |
| | protocol) | initiate | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| P | Processing | Tissue damage | incorrect diagnosis | 9 | Device malfunction | 1 | old, outdated equipment, | 1 |
| | (automated | | | | | | unreliable equipment, lack | |
| | processing) | | | | | | of maintenance logs, | |
| | | | | | | | expired service contracts, | |
| | | | | | | | lack of backup device, new | |
| | | | | | | | staff, high turnover in staff | |
| P | Processing | Tissue damage | rebiopsy | 3 | Device malfunction | 1 | old, outdated equipment, | 1 |
| | (automated | | | | | | unreliable equipment, lack | |
| | processing) | | | | | | of maintenance logs, | |
| | | | | | | | expired service contracts, | |
| | | | | | | | lack of backup device, new | |
| | | | | | | | staff, high turnover in staff | |
| P | Processing | Tissue damage | Delay in diagnosis | 3 | Device malfunction | 3 | old, outdated equipment, | 1 |
| | (automated | | | | | | unreliable equipment, lack | |
| | processing) | | | | | | of maintenance logs, | |
| | | | | | | | expired service contracts, | |
| | | | | | | | lack of backup device, new | |
| | | | | | | | staff, high turnover in staff | |
| E | Embedding (place | Tissue damage | incorrect diagnosis | 9 | Human error (Drop tissue, | 1 | lack of maintenance log, | 1 |
| | container in or | | | | used dirty transport | | dirty receptacles, wax | |
| | next to embedder) | | | | containers, place into dirty | | everywhere, visual clues, | |
| | | | | | receptacles, etc.) | | dirty blocks, sludge | |
| E | Embedding (place | Tissue damage | rebiopsy | 3 | Human error (Drop tissue, | 1 | lack of maintenance log, | 1 |
| | container in or | | | | used dirty transport | | dirty receptacles, wax | |
| | next to embedder) | | | | containers, place into dirty | | everywhere, visual clues, | |
| | | | | | receptacles, etc.) | | dirty blocks, sludge | |
| E | Embedding (place | Tissue damage | Delay in diagnosis | 3 | Human error (Drop tissue, | 3 | lack of maintenance log, | 1 |
| | container in or | | | | used dirty transport | | dirty receptacles, wax | |
| | next to embedder) | | | | containers, place into dirty | | everywhere, visual clues, | |
| | | | | | receptacles, etc.) | | dirty blocks, sludge | |
| E | Embedding (place | Loss of tissue | incorrect diagnosis | 9 | Human error (Drop tissue, | 1 | lack of maintenance log, | 1 |
| | container in or | | | | used dirty transport | | dirty receptacles, wax | |
| | next to embedder) | | | | containers, place into dirty | | everywhere, visual clues, | |
| | | | | | receptacles, etc.) | | dirty blocks, sludge | |
| E | Embedding (place | Loss of tissue | rebiopsy | 3 | Human error (Drop tissue, | 1 | lack of maintenance log, | 1 |
| | container in or | | | | used dirty transport | | dirty receptacles, wax | |
| | next to embedder) | | | | containers, place into dirty | | everywhere, visual clues, | |
| | | | | | receptacles, etc.) | | dirty blocks, sludge | |
| E | Embedding (place | Loss of tissue | Delay in diagnosis | 3 | Human error (Drop tissue, | 1 | lack of maintenance log, | 1 |
| | container in or | | | | used dirty transport | | dirty receptacles, wax | |
| | next to embedder) | | | | containers, place into dirty | | everywhere, visual clues, | |
| | | | | | receptacles, etc.) | | dirty blocks, sludge | |
| E | Embedding | Tissue damage | incorrect diagnosis | 9 | Human error (Drop tissue, | 1 | lack of maintenance log, | 1 |
| | (unload cassettes/ | | | | throwing away tissue, place | | dirty receptacles, wax | |
| | organize . . .) | | | | on dirty surface - i.e. | | everywhere, visual cues, | |
| | | | | | embedder, etc.) | | dirty blocks, sludge | |
| E | Embedding | Tissue damage | rebiopsy | 3 | Human error (Drop tissue, | 1 | lack of maintenance log, | 1 |
| | (unload cassettes/ | | | | throwing away tissue, place | | dirty receptacles, wax | |
| | organize . . .) | | | | on dirty surface - i.e. | | everywhere, visual cues, | |
| | | | | | embedder, etc.) | | dirty blocks, sludge | |
| E | Embedding | Tissue damage | Delay in diagnosis | 3 | Human error (Drop tissue, | 1 | lack of maintenance log, | 1 |
| | (unload cassettes/ | | | | throwing away tissue, place | | dirty receptacles, wax | |
| | organize . . .) | | | | on dirty surface - i.e. | | everywhere, visual cues, | |
| | | | | | embedder, etc.) | | dirty blocks, sludge | |
| E | Embedding | Loss of tissue | incorrect diagnosis | 9 | Human error (Drop tissue, | 1 | lack of maintenance log, | 1 |
| | (unload cassettes/ | | | | throwing away tissue, place | | dirty receptacles, wax | |
| | organize . . .) | | | | on dirty surface - i.e. | | everywhere, visual cues, | |
| | | | | | embedder, etc.) | | dirty blocks, sludge | |
| E | Embedding | Loss of tissue | rebiopsy | 3 | Human error (Drop tissue, | 1 | lack of maintenance log, | 1 |
| | (unload cassettes/ | | | | throwing away tissue, place | | dirty receptacles, wax | |
| | organize . . .) | | | | on dirty surface - i.e. | | everywhere, visual cues, | |
| | | | | | embedder, etc.) | | dirty blocks, sludge | |
| E | Embedding | Loss of tissue | Delay in diagnosis | 3 | Human error (Drop tissue, | 1 | lack of maintenance log, | 1 |
| | (unload cassettes/ | | | | throwing away tissue, place | | dirty receptacles, wax | |
| | organize . . .) | | | | on dirty surface - i.e. | | everywhere, visual cues, | |
| | | | | | embedder, etc.) | | dirty blocks, sludge | |
| E | Embedding (open, | Patient/data | incorrect diagnosis | 9 | Human error (Place incorrect | 1 | batch processing, multiple | 1 |
| | orient, fill with | mismatch | | | cassette onto tissue mold, | | cassettes out at office, messy | |
| | wax, cassette, fill, | | | | etc.) | | workspace | |
| | place on cold | | | | | | | |
| | plate) | | | | | | | |
| E | Embedding (open, | Patient/data | rebiopsy | 3 | Human error (Place incorrect | 1 | batch processing, multiple | 1 |
| | orient, fill with | mismatch | | | cassette onto tissue mold, | | cassettes out at office, messy | |
| | wax, cassette, fill, | | | | etc.) | | workspace | |
| | place on cold | | | | | | | |
| | plate) | | | | | | | |
| E | Embedding (open, | Patient/data | Delay in diagnosis | 3 | Human error (Place incorrect | 1 | batch processing, multiple | 1 |
| | orient, fill with | mismatch | | | cassette onto tissue mold, | | cassettes out at office, messy | |
| | wax, cassette, fill, | | | | etc.) | | workspace | |
| | place on cold | | | | | | | |
| | plate) | | | | | | | |
| E | Embedding | Loss of tissue | incorrect diagnosis | 9 | Human error (Throw away | 1 | Lack of specimen tracking, | 1 |
| | (reconcile and | | | | tissue stuck to mold, drop | | batch processing, messy | |
| | transport to | | | | tissue, allocate to incorrect | | workstation, lack of visual | |
| | microtomy) | | | | station/pathologist, etc.) | | cues, lack of identified | |
| | | | | | | | tissue cassettes | |
| E | Embedding | Loss of tissue | rebiopsy | 3 | Human error (Throw away | 1 | Lack of specimen tracking, | 1 |
| | (reconcile and | | | | tissue stuck to mold, drop | | batch processing, messy | |
| | transport to | | | | tissue, allocate to incorrect | | workstation, lack of visual | |
| | microtomy) | | | | station/pathologist, etc.) | | cues, lack of identified | |
| | | | | | | | tissue cassettes | |
| E | Embedding | Loss of tissue | Delay in diagnosis | 3 | Human error (Throw away | 1 | Lack of specimen tracking, | 1 |
| | (reconcile and | | | | tissue stuck to mold, drop | | batch processing, messy | |
| | transport to | | | | tissue, allocate to incorrect | | workstation, lack of visual | |
| | microtomy) | | | | station/pathologist, etc.) | | cues, lack of identified | |
| | | | | | | | tissue cassettes | |
| E | Unloading/ | Tissue damage | Delay in diagnosis | 3 | Human error (Forget tissue, | 1 | Lack of specimen tracking, | 1 |
| | Transport to | | | | drop tissue, forget to obtain | | batch processing, messy | |
| | microtomy | | | | tissue, leave in embedder) | | workstation, lack of visual | |
| | | | | | | | cues, lack of identified | |
| | | | | | | | tissue cassettes | |
| E | Unloading/ | Tissue damage | Rebiopsy | 3 | Human error (Forget tissue, | 1 | Lack of specimen tracking, | 1 |
| | Transport to | | | | drop tissue, forget to obtain | | batch processing, messy | |
| | microtomy | | | | tissue, leave in embedder) | | workstation, lack of visual | |
| | | | | | | | cues, lack of identified | |
| | | | | | | | tissue cassettes | |
| E | Unloading/ | Loss of tissue | Incorrect | 9 | Human error (Forget tissue, | 1 | Lack of specimen tracking, | 1 |
| | Transport to | | Diagnosis | | drop tissue, forget to obtain | | batch processing, messy | |
| | microtomy | | | | tissue, leave in embedder) | | workstation, lack of visual | |
| | | | | | | | cues, lack of identified | |
| | | | | | | | tissue cassettes | |
| E | Unloading/ | Loss of tissue | Rebiopsy | 3 | Human error (Forget tissue, | 1 | Lack of specimen tracking, | 1 |
| | Transport to | | Diagnosis | | drop tissue, forget to obtain | | batch processing, messy | |
| | microtomy | | | | tissue, leave in embedder) | | workstation, lack of visual | |
| | | | | | | | cues, lack of identified | |
| | | | | | | | tissue cassettes | |
| M | Microtomy | Tissue damage | incorrect diagnosis | 9 | Human error (face too deep, | 1 | dark environment, small | 1 |
| | (unpack, sort, trim | | | | cut tissue, damage tissue, | | tissue, lack of experience, | |
| | and face blocks) | | | | etc.) | | non-automated microtome | |
| M | Microtomy | Tissue damage | rebiopsy | 3 | Human error (face too deep, | 1 | dark environment, small | 1 |
| | (unpack, sort, trim | | | | cut tissue, damage tissue, | | tissue, lack of experience, | |
| | and face blocks) | | | | etc.) | | non-automated microtome | |
| M | Microtomy | Tissue damage | Delay in diagnosis | 3 | Human error (face too deep, | 1 | dark environment, small | 1 |
| | (unpack, sort, trim | | | | cut tissue, damage tissue, | | tissue, lack of experience, | |
| | and face blocks) | | | | etc.) | | non-automated microtome | |
| M | Microtomy | Loss of tissue | incorrect diagnosis | 9 | Human error (throw block | 1 | Full trash, new staff, lack of | 1 |
| | (unpack, sort, trim | | | | away, etc.) | | experience, batch | |
| | and face blocks) | | | | | | processing, dirty lab, high | |
| | | | | | | | throughput | |
| M | Microtomy | Loss of tissue | rebiopsy | 3 | Human error (throw block | 1 | Full trash, new staff, lack of | 1 |
| | (unpack, sort, trim | | | | away, etc.) | | experience, batch | |
| | and face blocks) | | | | | | processing, dirty lab, high | |
| | | | | | | | throughput | |
| M | Microtomy | Loss of tissue | Delay in diagnosis | 3 | Human error (throw block | 1 | Full trash, new staff, lack of | 1 |
| | (unpack, sort, trim | | | | away, etc.) | | experience, batch | |
| | and face blocks) | | | | | | processing, dirty lab, high | |
| | | | | | | | throughput | |
| M | Microtomy (ice) | Tissue damage | incorrect diagnosis | 9 | Human error (left cassette on | 1 | Batch processing, high | 1 |
| | | | | | ice too long, left cassette on | | volumes, full ice trays (with | |
| | | | | | ice too short, etc.) | | cassettes), melting ice | |
| | | | | | | | blocks, processing artifacts, | |
| | | | | | | | sunken blocks after storage | |
| M | Microtomy (ice) | Tissue damage | rebiopsy | 3 | Human error (left cassette on | 1 | Batch processing, high | 1 |
| | | | | | ice too long, left cassette on | | volumes, full ice trays (with | |
| | | | | | ice too short, etc.) | | cassettes), melting ice | |
| | | | | | | | blocks, processing artifacts, | |
| | | | | | | | sunken blocks after storage | |
| M | Microtomy (ice) | Tissue damage | Delay in diagnosis | 3 | Human error (left cassette on | 1 | Batch processing, high | 1 |
| | | | | | ice too long, left cassette on | | volumes, full ice trays (with | |
| | | | | | ice too short, etc.) | | cassettes), melting ice | |
| | | | | | | | blocks, processing artifacts, | |
| | | | | | | | sunken blocks after storage | |
| M | Microtomy (ice) | Loss of tissue | incorrect diagnosis | 9 | Human error (left cassette on | 1 | Batch processing, high | 1 |
| | | | | | ice too long, left cassette on | | volumes, full ice trays (with | |
| | | | | | ice too short, etc.) | | cassettes), melting ice | |
| | | | | | | | blocks, processing artifacts, | |
| | | | | | | | sunken blocks after storage | |
| M | Microtomy (ice) | Loss of tissue | rebiopsy | 3 | Human error (left cassette on | 1 | Batch processing, high | 1 |
| | | | | | ice too long, left cassette on | | volumes, full ice trays (with | |
| | | | | | ice too short, etc.) | | cassettes), melting ice | |
| | | | | | | | blocks, processing artifacts, | |
| | | | | | | | sunken blocks after storage | |
| M | Microtomy (ice) | Loss of tissue | Delay in diagnosis | 3 | Human error (left cassette on | 1 | Batch processing, high | 1 |
| | | | | | ice too long, left cassette on | | volumes, full ice trays (with | |
| | | | | | ice too short, etc.) | | cassettes), melting ice | |
| | | | | | | | blocks, processing artifacts, | |
| | | | | | | | sunken blocks after storage | |
| M | Microtomy | Patient/data | incorrect diagnosis | 9 | Human error (transpose, | 3 | batch processing, multiple | 1 |
| | (matching, printing | mismatch | | | wrong side, wrong block, | | consumables out at once, | |
| | slide, finding slide, | | | | omit slide, omit block, | | messy workspace, high | |
| | etc.) | | | | selected incorrect test panel, | | volume, lack of specimen | |
| | | | | | incorrect #slides pulled, etc.) | | tracking | |
| M | Microtomy | Patient/data | rebiopsy | 3 | Human error (transpose, | 1 | batch processing, multiple | 1 |
| | (matching, printing | mismatch | | | wrong side, wrong block, | | consumables out at once, | |
| | slide, finding slide, | | | | omit slide, omit block, | | messy workspace, high | |
| | etc.) | | | | selected incorrect test panel, | | volume, lack of specimen | |
| | | | | | incorrect #slides pulled, etc.) | | tracking | |
| M | Microtomy | Patient/data | Delay in diagnosis | 3 | Human error (transpose, | 3 | batch processing, multiple | 1 |
| | (matching, printing | mismatch | | | wrong side, wrong block, | | consumables out at once, | |
| | slide, finding slide, | | | | omit slide, omit block, | | messy workspace, high | |
| | etc.) | | | | selected incorrect test panel, | | volume, lack of specimen | |
| | | | | | incorrect #slides pulled, etc.) | | tracking | |
| M | Microtomy | Tissue damage | incorrect diagnosis | 9 | Human error (machine set up, | 1 | dark environment, small | 1 |
| | (cutting) | | | | using dull blades, wrong | | tissue, lack of experience, | |
| | | | | | consumables, wrong settings, | | non-automated microtome | |
| | | | | | lack of maintenance, angles, | | | |
| | | | | | cut away tissue, trim through | | | |
| | | | | | block, wrong orientation, out | | | |
| | | | | | of plane, etc.) | | | |
| M | Microtomy | Tissue damage | rebiopsy | 3 | Human error (machine set up, | 1 | dark environment, small | 1 |
| | (cutting) | | | | using dull blades, wrong | | tissue, lack of experience, | |
| | | | | | consumables, wrong settings, | | non-automated microtome | |
| | | | | | lack of maintenance, angles, | | | |
| | | | | | cut away tissue, trim through | | | |
| | | | | | block, wrong orientation, out | | | |
| | | | | | of plane, etc.) | | | |
| M | Microtomy | Tissue damage | Delay in diagnosis | 3 | Human error (machine set up, | 3 | dark environment, small | 1 |
| | (cutting) | | | | using dull blades, wrong | | tissue, lack of experience, | |
| | | | | | consumables, wrong settings, | | non-automated microtome | |
| | | | | | lack of maintenance, angles, | | | |
| | | | | | cut away tissue, trim through | | | |
| | | | | | block, wrong orientation, out | | | |
| | | | | | of plane, etc.) | | | |
| M | Microtomy | Loss of tissue | incorrect diagnosis | 9 | Human error (machine set up, | 3 | dark environment, small | 1 |
| | (cutting) | | | | using dull blades, wrong | | tissue, lack of experience, | |
| | | | | | consumables, wrong settings, | | non-automated microtome | |
| | | | | | lack of maintenance, angles, | | | |
| | | | | | cut away tissue, trim through | | | |
| | | | | | block, wrong orientation, out | | | |
| | | | | | of plane, etc.) | | | |
| M | Microtomy | Loss of tissue | rebiopsy | 3 | Human error (machine set up, | 1 | dark environment, small | 1 |
| | (cutting) | | | | using dull blades, wrong | | tissue, lack of experience, | |
| | | | | | consumables, wrong settings, | | non-automated microtome | |
| | | | | | lack of maintenance, angles, | | | |
| | | | | | cut away tissue, trim through | | | |
| | | | | | block, wrong orientation, out | | | |
| | | | | | of plane, etc.) | | | |
| M | Microtomy | Loss of tissue | Delay in diagnosis | 3 | Human error (machine set up, | 3 | dark environment, small | 1 |
| | (cutting) | | | | using dull blades, wrong | | tissue, lack of experience, | |
| | | | | | consumables, wrong settings, | | non-automated microtome | |
| | | | | | lack of maintenance, angles, | | | |
| | | | | | cut away tissue, trim through | | | |
| | | | | | block, wrong orientation, out | | | |
| | | | | | of plane, etc.) | | | |
| M | Microtomy | Tissue damage | incorrect diagnosis | 9 | Device malfunction | 1 | old, outdated equipment, | 1 |
| | (cutting) | | | | | | unreliable equipment, lack | |
| | | | | | | | of maintenance logs | |
| | | | | | | | expired service contracts, | |
| | | | | | | | lack of backup device, new | |
| | | | | | | | staff, high turnover in staff | |
| M | Microtomy | Tissue damage | rebiopsy | 3 | Device malfunction | 1 | old, outdated equipment, | 1 |
| | (cutting) | | | | | | unreliable equipment, lack | |
| | | | | | | | of maintenance logs | |
| | | | | | | | expired service contracts, | |
| | | | | | | | lack of backup device, new | |
| | | | | | | | staff, high turnover in staff | |
| M | Microtomy | Tissue damage | Delay in diagnosis | 3 | Device malfunction | 1 | old, outdated equipment, | 1 |
| | (cutting) | | | | | | unreliable equipment, lack | |
| | | | | | | | of maintenance logs | |
| | | | | | | | expired service contracts, | |
| | | | | | | | lack of backup device, new | |
| | | | | | | | staff, high turnover in staff | |
| M | Microtomy | Loss of tissue | incorrect diagnosis | 9 | Device malfunction | 1 | old, outdated equipment, | 1 |
| | (cutting) | | | | | | unreliable equipment, lack | |
| | | | | | | | of maintenance logs | |
| | | | | | | | expired service contracts, | |
| | | | | | | | lack of backup device, new | |
| | | | | | | | staff, high turnover in staff | |
| M | Microtomy | Loss of tissue | rebiopsy | 3 | Device malfunction | 1 | old, outdated equipment, | 1 |
| | (cutting) | | | | | | unreliable equipment, lack | |
| | | | | | | | of maintenance logs | |
| | | | | | | | expired service contracts, | |
| | | | | | | | lack of backup device, new | |
| | | | | | | | staff, high turnover in staff | |
| M | Microtomy | Loss of tissue | Delay in diagnosis | 3 | Device malfunction | 1 | old, outdated equipment, | 1 |
| | (cutting) | | | | | | unreliable equipment, lack | |
| | | | | | | | of maintenance logs | |
| | | | | | | | expired service contracts, | |
| | | | | | | | lack of backup device, new | |
| | | | | | | | staff, high turnover in staff | |
| M | Microtomy | Loss of tissue | incorrect diagnosis | 9 | Human error (choice of tools | 1 | new staff, cleanliness, | 1 |
| | (transfer to | | | | used, miss water bath, stick it | | tissue stuck in places it | |
| | waterbath) | | | | somewhere unintentionally, | | shouldn't be stuck to | |
| | | | | | etc.) | | | |
| M | Microtomy | Loss of tissue | rebiopsy | 3 | Human error (choice of tools | 1 | new staff, cleanliness, | 1 |
| | (transfer to | | | | used, miss water bath, stick it | | tissue stuck in places it | |
| | waterbath) | | | | somewhere unintentionally, | | shouldn't be stuck to | |
| | | | | | etc.) | | | |
| M | Microtomy | Loss of tissue | Delay in diagnosis | 3 | Human error (choice of tools | 1 | new staff, cleanliness, | 1 |
| | (transfer to | | | | used, miss water bath, stick it | | tissue stuck in places it | |
| | waterbath) | | | | somewhere unintentionally, | | shouldn't be stuck to | |
| | | | | | etc.) | | | |
| M | Microtomy | Tissue damage | incorrect diagnosis | 9 | Human error (too hot, too | 1 | dirty bath, lack of | 1 |
| | (prepare and using | | | | cold, not enough H2O, too | | thermometer, lack of | |
| | water bath) | | | | much H2O, contaminated | | temperature gauge, | |
| | | | | | water, in too long, not long | | additives out next to | |
| | | | | | enough, additives, etc.) | | waterbath, high volume, | |
| | | | | | | | batch processing | |
| M | Microtomy | Tissue damage | rebiopsy | 3 | Human error (too hot, too | 1 | dirty bath, lack of | 1 |
| | (prepare and using | | | | cold, not enough H2O, too | | thermometer, lack of | |
| | water bath) | | | | much H2O, contaminated | | temperature gauge, | |
| | | | | | water, in too long, not long | | additives out next to | |
| | | | | | enough, additives, etc.) | | waterbath, high volume, | |
| | | | | | | | batch processing | |
| M | Microtomy | Tissue damage | Delay in diagnosis | 3 | Human error (too hot, too | 3 | dirty bath, lack of | 1 |
| | (prepare and using | | | | cold, not enough H2O, too | | thermometer, lack of | |
| | water bath) | | | | much H2O, contaminated | | temperature gauge, | |
| | | | | | water, in too long, not long | | additives out next to | |
| | | | | | enough, additives, etc.) | | waterbath, high volume, | |
| | | | | | | | batch processing | |
| M | Microtomy | Loss of tissue | incorrect diagnosis | 9 | Human error (too hot, too | 1 | dirty bath, lack of | 1 |
| | (prepare and using | | | | cold, not enough H2O, too | | thermometer, lack of | |
| | water bath) | | | | much H2O, contaminated | | temperature gauge, | |
| | | | | | water, in too long, not long | | additives out next to | |
| | | | | | enough, additives, etc.) | | waterbath, high volume, | |
| | | | | | | | batch processing | |
| M | Microtomy | Loss of tissue | rebiopsy | 3 | Human error (too hot, too | 1 | dirty bath, lack of | 1 |
| | (prepare and using | | | | cold, not enough H2O, too | | thermometer, lack of | |
| | water bath) | | | | much H2O, contaminated | | temperature gauge, | |
| | | | | | water, in too long, not long | | additives out next to | |
| | | | | | enough, additives, etc.) | | waterbath, high volume, | |
| | | | | | | | batch processing | |
| M | Microtomy | Loss of tissue | Delay in diagnosis | 3 | Human error (too hot, too | 3 | dirty bath, lack of | 1 |
| | (prepare and using | | | | cold, not enough H2O, too | | thermometer, lack of | |
| | water bath) | | | | much H2O, contaminated | | temperature gauge, | |
| | | | | | water, in too long, not long | | additives out next to | |
| | | | | | enough, additives, etc.) | | waterbath, high volume, | |
| | | | | | | | batch processing | |
| M | Microtomy | Tissue damage | incorrect diagnosis | 9 | Device malfunction | 1 | old, outdated equipment, | 1 |
| | (prepare and using | | | | | | unreliable equipment, lack | |
| | water bath) | | | | | | of maintenance logs, | |
| | | | | | | | expired service contracts, | |
| | | | | | | | lack of backup device, new | |
| | | | | | | | staff, high turnover in staff | |
| M | Microtomy | Tissue damage | rebiopsy | 3 | Device malfunction | 1 | old, outdated equipment, | 1 |
| | (prepare and using | | | | | | unreliable equipment, lack | |
| | water bath) | | | | | | of maintenance logs, | |
| | | | | | | | expired service contracts, | |
| | | | | | | | lack of backup device, new | |
| | | | | | | | staff, high turnover in staff | |
| M | Microtomy | Tissue damage | Delay in diagnosis | 3 | Device malfunction | 1 | old, outdated equipment, | 1 |
| | (prepare and using | | | | | | unreliable equipment, lack | |
| | water bath) | | | | | | of maintenance logs, | |
| | | | | | | | expired service contracts, | |
| | | | | | | | lack of backup device, new | |
| | | | | | | | staff, high turnover in staff | |
| M | Microtomy | Loss of tissue | incorrect diagnosis | 9 | Device malfunction | 1 | old, outdated equipment, | 1 |
| | (prepare and using | | | | | | unreliable equipment, lack | |
| | water bath) | | | | | | of maintenance logs, | |
| | | | | | | | expired service contracts, | |
| | | | | | | | lack of backup device, new | |
| | | | | | | | staff, high turnover in staff | |
| M | Microtomy | Loss of tissue | rebiopsy | 3 | Device malfunction | 1 | old, outdated equipment, | 1 |
| | (prepare and using | | | | | | unreliable equipment, lack | |
| | water bath) | | | | | | of maintenance logs, | |
| | | | | | | | expired service contracts, | |
| | | | | | | | lack of backup device, new | |
| | | | | | | | staff, high turnover in staff | |
| M | Microtomy | Loss of tissue | Delay in diagnosis | 3 | Device malfunction | 1 | old, outdated equipment, | 1 |
| | (prepare and using | | | | | | unreliable equipment, lack | |
| | water bath) | | | | | | of maintenance logs, | |
| | | | | | | | expired service contracts, | |
| | | | | | | | lack of backup device, new | |
| | | | | | | | staff, high turnover in staff | |
| M | Microtomy | Tissue damage | incorrect diagnosis | 9 | Human error (orientation, | 1 | new staff, high volume, | 1 |
| | (transfer from | | | | incorrect selection, add | | waterbath is full, dirty | |
| | water bath to slide | | | | artifact, contaminate, dirty | | water, batch processing, | |
| | and into slide rack) | | | | slides, incorrect consumables, | | additives sitting out, poor | |
| | | | | | incomplete drying (i.e. flick | | processing, artifact in final | |
| | | | | | water off), etc.) | | slides | |
| M | Microtomy | Tissue damage | rebiopsy | 3 | Human error (orientation, | 1 | new staff, high volume, | 1 |
| | (transfer from | | | | incorrect selection, add | | waterbath is full, dirty | |
| | water bath to slide | | | | artifact, contaminate, dirty | | water, batch processing, | |
| | and into slide rack) | | | | slides, incorrect consumables, | | additives sitting out, poor | |
| | | | | | incomplete drying (i.e. flick | | processing, artifact in final | |
| | | | | | water off), etc.) | | slides | |
| M | Microtomy | Tissue damage | Delay in diagnosis | 3 | Human error (orientation, | 1 | new staff, high volume, | 1 |
| | (transfer from | | | | incorrect selection, add | | waterbath is full, dirty | |
| | water bath to slide | | | | artifact, contaminate, dirty | | water, batch processing, | |
| | and into slide rack) | | | | slides, incorrect consumables, | | additives sitting out, poor | |
| | | | | | incomplete drying (i.e. flick | | processing, artifact in final | |
| | | | | | water off), etc.) | | slides | |
| M | Microtomy | Loss of tissue | incorrect diagnosis | 9 | Human error (orientation, | 1 | new staff, high volume, | 1 |
| | (transfer from | | | | incorrect selection, add | | waterbath is full, dirty | |
| | water bath to slide | | | | artifact, contaminate, dirty | | water, batch processing, | |
| | and into slide rack) | | | | slides, incorrect consumables, | | additives sitting out, poor | |
| | | | | | incomplete drying (i.e. flick | | processing, artifact in final | |
| | | | | | water off), etc.) | | slides | |
| M | Microtomy | Loss of tissue | rebiopsy | 3 | Human error (orientation, | 1 | new staff, high volume, | 1 |
| | (transfer from | | | | incorrect selection, add | | waterbath is full, dirty | |
| | water bath to slide | | | | artifact, contaminate, dirty | | water, batch processing, | |
| | and into slide rack) | | | | slides, incorrect consumables, | | additives sitting out, poor | |
| | | | | | incomplete drying (i.e. flick | | processing, artifact in final | |
| | | | | | water off), etc.) | | slides | |
| M | Microtomy | Loss of tissue | Delay in diagnosis | 3 | Human error (orientation, | 3 | new staff, high volume, | 1 |
| | (transfer from | | | | incorrect selection, add | | waterbath is full, dirty | |
| | water bath to slide | | | | artifact, contaminate, dirty | | water, batch processing, | |
| | and into slide rack) | | | | slides, incorrect consumables, | | additives sitting out, poor | |
| | | | | | incomplete drying (i.e. flick | | processing, artifact in final | |
| | | | | | water off), etc.) | | slides | |
| M | Microtomy | patient/data | incorrect diagnosis | 9 | Human error (tissue placed | 3 | batch processing, high | 1 |
| | (transfer from | mismatch | | | onto wrong slide) | | volumes, lack of specimen | |
| | water bath to slide | | | | | | tracking, batch slides | |
| | and into slide rack) | | | | | | printed out, stacks of slides, | |
| | | | | | | | handwritten slides, etc. | |
| M | Microtomy | patient/data | rebiopsy | 3 | Human error (tissue placed | 1 | batch processing, high | 1 |
| | (transfer from | mismatch | | | onto wrong slide) | | volumes, lack of specimen | |
| | water bath to slide | | | | | | tracking, batch slides | |
| | and into slide rack) | | | | | | printed out, stacks of slides, | |
| | | | | | | | handwritten slides, etc. | |
| M | Microtomy | patient/data | Delay in diagnosis | 3 | Human error (tissue placed | 9 | batch processing, high | 1 |
| | (transfer from | mismatch | | | onto wrong slide) | | volumes, lack of specimen | |
| | water bath to slide | | | | | | tracking, batch slides | |
| | and into slide rack) | | | | | | printed out, stacks of slides, | |
| | | | | | | | handwritten slides, etc. | |
| Stain | Dry slides | tissue damage | incorrect diagnosis | 9 | Human error (too long, too | 1 | Lack of thermometers, | 1 |
| | | | | | short, skip step, too hot, too | | timers, poor maintenance, | |
| | | | | | cold, etc.) | | lack of logs, batch | |
| | | | | | | | processing, high volume/ | |
| | | | | | | | low time, etc. | |
| Stain | Dry slides | tissue damage | rebiopsy | 3 | Human error (too long, too | 1 | Lack of thermometers, | 1 |
| | | | | | short, skip step, too hot, too | | timers, poor maintenance, | |
| | | | | | cold, etc.) | | lack of logs, batch | |
| | | | | | | | processing, high volume/ | |
| | | | | | | | low time, etc. | |
| Stain | Dry slides | tissue damage | Delay in diagnosis | 3 | Human error (too long, too | 3 | Lack of thermometers, | 1 |
| | | | | | short, skip step, too hot, too | | timers, poor maintenance, | |
| | | | | | cold, etc.) | | lack of logs, batch | |
| | | | | | | | processing, high volume/ | |
| | | | | | | | low time, etc. | |
| Stain | Primary Staining | Tissue damage | incorrect diagnosis | 9 | Human error (drop slides | 1 | Batch processing, poor | 1 |
| | (load stainer) | | | | inside stain container, not seat | | practice, large volume in | |
| | | | | | properly, improper | | limited time, multi-platform | |
| | | | | | positioning, using wrong | | rack/utilization, multiple | |
| | | | | | rack) | | stainer types | |
| Stain | Primary Staining | Tissue damage | rebiopsy | 3 | Human error (drop slides | 1 | Batch processing, poor | 1 |
| | (load stainer) | | | | inside stain container, not seat | | practice, large volume in | |
| | | | | | properly, improper | | limited time, multi-platform | |
| | | | | | positioning, using wrong | | rack/utilization, multiple | |
| | | | | | rack) | | stainer types | |
| Stain | Primary Staining | Tissue damage | Delay in diagnosis | 3 | Human error (drop slides | 1 | Batch processing, poor | 1 |
| | (load stainer) | | | | inside stain container, not seat | | practice, large volume in | |
| | | | | | properly, improper | | limited time, multi-platform | |
| | | | | | positioning, using wrong | | rack/utilization, multiple | |
| | | | | | rack) | | stainer types | |
| Stain | Primary Staining | Loss of tissue | incorrect diagnosis | 9 | Human error (Drying time | 3 | Batch processing, poor | 3 |
| | (load stainer) | | | | too short, underprocessed | | practice, large volume in | |
| | | | | | tissue, inadequate processing | | limited time, multi-platform | |
| | | | | | for tissue type, incorrect slide | | rack/utilization, multiple | |
| | | | | | type - charged vs. non- | | stainer types | |
| | | | | | charged, drop slides inside | | | |
| | | | | | staining container, not seat | | | |
| | | | | | properly, improper | | | |
| | | | | | positioning, using wrong | | | |
| | | | | | rack) | | | |
| Stain | Primary Staining | Loss of tissue | rebiopsy | 3 | Human error (Drying time | 3 | Batch processing, poor | 3 |
| | (load stainer) | | | | too short, underprocessed | | practice, large volume in | |
| | | | | | tissue, inadequate processing | | limited time, multi-platform | |
| | | | | | for tissue type, incorrect slide | | rack/utilization, multiple | |
| | | | | | type - charged vs. non- | | stainer types | |
| | | | | | charged, drop slides inside | | | |
| | | | | | staining container, not seat | | | |
| | | | | | properly, improper | | | |
| | | | | | positioning, using wrong | | | |
| | | | | | rack) | | | |
| Stain | Primary Staining | Loss of tissue | Delay in diagnosis | 3 | Human error (Drying time | 3 | Batch processing, poor | 3 |
| | (load stainer) | | | | too short, underprocessed | | practice, large volume in | |
| | | | | | tissue, inadequate processing | | limited time, multi-platform | |
| | | | | | for tissue type, incorrect slide | | rack/utilization, multiple | |
| | | | | | type - charged vs. non- | | stainer types | |
| | | | | | charged, drop slides inside | | | |
| | | | | | staining container, not seat | | | |
| | | | | | properly, improper | | | |
| | | | | | positioning, using wrong | | | |
| | | | | | rack) | | | |
| Stain | Primary Staining | Processor won't | Delay in diagnosis | 3 | Human error (don't close | 1 | Batch processing, poor | 1 |
| | (load stainer) | initiate (as result | | | door, forgot to load, input | | practice, large volume in | |
| | | of human error) | | | errors, ordering errors, etc.) | | limited time, multi-platform | |
| | | | | | | | rack/utilization, multiple | |
| | | | | | | | stainer types | |
| Stain | Primary Staining | Tissue damage | incorrect diagnosis | 9 | Human error (wrong | 1 | Batch processing, poor | 1 |
| | (initiate protocol) | | | | protocol, wrong rack, edit | | practice, large volume in | |
| | | | | | incorrectly/override, | | limited time, multi-platform | |
| | | | | | override warnings, ignore | | rack/utilization, multiple | |
| | | | | | user actions, etc.) | | stainer types | |
| Stain | Primary Staining | Tissue damage | rebiopsy | 3 | Human error (wrong | 1 | Batch processing, poor | 1 |
| | (initiate protocol) | | | | protocol, wrong rack, edit | | practice, large volume in | |
| | | | | | incorrectly/override, | | limited time, multi-platform | |
| | | | | | override warnings, ignore | | rack/utilization, multiple | |
| | | | | | user actions, etc.) | | stainer types | |
| Stain | Primary Staining | Tissue damage | Delay in diagnosis | 3 | Human error (wrong | 3 | Batch processing, poor | 1 |
| | (initiate protocol) | | | | protocol, wrong rack, edit | | practice, large volume in | |
| | | | | | incorrectly/override, | | limited time, multi-platform | |
| | | | | | override warnings, ignore | | rack/utilization, multiple | |
| | | | | | user actions, etc.) | | stainer types | |
| Stain | Primary Staining | Tissue damage | incorrect diagnosis | 9 | Device malfunction | 1 | old, outdated equipment, | 1 |
| | (initiate protocol) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| Stain | Primary Staining | Tissue damage | rebiopsy | 3 | Device malfunction | 1 | old, outdated equipment, | 1 |
| | (initiate protocol) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| Stain | Primary Staining | Tissue damage | Delay in diagnosis | 3 | Device malfunction | 1 | old, outdated equipment, | 1 |
| | (initiate protocol) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| Stain | Primary Staining | Stainer won't | Delay in diagnosis | 3 | Human error (forgot to hit | 1 | Batch processing, poor | 1 |
| | (initiate protocol) | initiate (as result | | | ″start″, forgot to close door, | | practice, large volume in | |
| | | of human error) | | | keep lids on, etc.) | | limited time, multi-platform | |
| | | | | | | | rack/utilization, multiple | |
| | | | | | | | stainer types | |
| Stain | Primary Staining | Stainer won't | Delay in diagnosis | 3 | Device malfunction | 1 | old, outdated equipment, | 1 |
| | (initiate protocol) | initiate | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| Stain | Primary Staining | Loss of tissue | incorrect diagnosis | 9 | Human error (Drying time | 3 | Multiple hand-offs within | 1 |
| | (automated | | | | too short, underprocessed | | departments, lack of timers, | |
| | staining) | | | | tissue, inadequate processing | | new staff, lack of specimen | |
| | | | | | for tissue type, incorrect slide | | tracking, low experience, | |
| | | | | | type - charged vs. non- | | numerous slide types, poor | |
| | | | | | charged, poor sectioning, | | procedures, sunken blocks, | |
| | | | | | etc.) | | messy lab, multiple | |
| | | | | | | | instruments cross vendors | |
| Stain | Primary Staining | Loss of tissue | rebiopsy | 3 | Human error (Drying time | 3 | Multiple hand-offs within | 1 |
| | (automated | | | | too short, underprocessed | | departments, lack of timers, | |
| | staining) | | | | tissue, inadequate processing | | new staff, lack of specimen | |
| | | | | | for tissue type, incorrect slide | | tracking, low experience, | |
| | | | | | type - charged vs. non- | | numerous slide types, poor | |
| | | | | | charged, poor sectioning, | | procedures, sunken blocks, | |
| | | | | | etc.) | | messy lab, multiple | |
| | | | | | | | instruments cross vendors | |
| Stain | Primary Staining | Loss of tissue | Delay in diagnosis | 3 | Human error (Drying time | 3 | Multiple hand-offs within | 1 |
| | (automated | | | | too short, underprocessed | | departments, lack of timers, | |
| | staining) | | | | tissue, inadequate processing | | new staff, lack of specimen | |
| | | | | | for tissue type, incorrect slide | | tracking, low experience, | |
| | | | | | type - charged vs. non- | | numerous slide types, poor | |
| | | | | | charged, poor sectioning, | | procedures, sunken blocks, | |
| | | | | | etc.) | | messy lab, multiple | |
| | | | | | | | instruments cross vendors | |
| Stain | Primary Staining | cross | incorrect diagnosis | 9 | Human error (Stainer used for | 1 | Multiple hand-offs within | 1 |
| | (automated | contamination | | | cytology and not cleaned, | | departments, lack of timers, | |
| | staining) | | | | Drying time too short, | | new staff, lack of specimen | |
| | | | | | underprocessed tissue, | | tracking, low experience, | |
| | | | | | inadequate processing for | | numerous slide types, poor | |
| | | | | | tissue type, incorrect slide | | procedures, sunken blocks, | |
| | | | | | type - charged vs. non- | | messy lab, multiple | |
| | | | | | charged, poor sectioning, | | instruments cross vendors | |
| | | | | | etc.) | | | |
| Stain | Primary Staining | cross | rebiopsy | 3 | Human error (Stainer used for | 1 | Multiple hand-offs within | 1 |
| | (automated | contamination | | | cytology and not cleaned, | | departments, lack of timers, | |
| | staining) | | | | Drying time too short, | | new staff, lack of specimen | |
| | | | | | underprocessed tissue, | | tracking, low experience, | |
| | | | | | inadequate processing for | | numerous slide types, poor | |
| | | | | | tissue type, incorrect slide | | procedures, sunken blocks, | |
| | | | | | type - charged vs. non- | | messy lab, multiple | |
| | | | | | charged, poor sectioning, | | instruments cross vendors | |
| | | | | | etc.) | | | |
| Stain | Primary Staining | cross | Delay in diagnosis | 3 | Human error (Stainer used for | 1 | Multiple hand-offs within | 1 |
| | (automated | contamination | | | cytology and not cleaned, | | departments, lack of timers, | |
| | staining) | | | | Drying time too short, | | new staff, lack of specimen | |
| | | | | | underprocessed tissue, | | tracking, low experience, | |
| | | | | | inadequate processing for | | numerous slide types, poor | |
| | | | | | tissue type, incorrect slide | | procedures, sunken blocks, | |
| | | | | | type - charged vs. non- | | messy lab, multiple | |
| | | | | | charged, poor sectioning, | | instruments cross vendors | |
| | | | | | etc.) | | | |
| Stain | Primary Staining | Tissue damage | incorrect diagnosis | 9 | Device malfunction | 1 | old, outdated equipment, | 1 |
| | (automated | | | | | | unreliable equipment, lack | |
| | staining) | | | | | | of maintenance logs, | |
| | | | | | | | expired service contracts, | |
| | | | | | | | lack of backup device, new | |
| | | | | | | | staff, high turnover in staff | |
| Stain | Primary Staining | Tissue damage | rebiopsy | 3 | Device malfunction | 1 | old, outdated equipment, | 1 |
| | (automated | | | | | | unreliable equipment, lack | |
| | staining) | | | | | | of maintenance logs, | |
| | | | | | | | expired service contracts, | |
| | | | | | | | lack of backup device, new | |
| | | | | | | | staff, high turnover in staff | |
| Stain | Primary Staining | Tissue damage | Delay in diagnosis | 3 | Device malfunction | 1 | old, outdated equipment, | 1 |
| | (automated | | | | | | unreliable equipment, lack | |
| | staining) | | | | | | of maintenance logs, | |
| | | | | | | | expired service contracts, | |
| | | | | | | | lack of backup device, new | |
| | | | | | | | staff, high turnover in staff | |
| Stain | Primary Staining | Loss of tissue | incorrect diagnosis | 9 | Device malfunction | 1 | old, outdated equipment, | 1 |
| | (automated | | | | | | unreliable equipment, lack | |
| | staining) | | | | | | of maintenance logs, | |
| | | | | | | | expired service contracts, | |
| | | | | | | | lack of backup device, new | |
| | | | | | | | staff, high turnover in staff | |
| Stain | Primary Staining | Loss of tissue | rebiopsy | 3 | Device malfunction | 1 | old, outdated equipment, | 1 |
| | (automated | | | | | | unreliable equipment, lack | |
| | staining) | | | | | | of maintenance logs, | |
| | | | | | | | expired service contracts, | |
| | | | | | | | lack of backup device, new | |
| | | | | | | | staff, high turnover in staff | |
| Stain | Primary Staining | Loss of tissue | Delay in diagnosis | 3 | Device malfunction | 1 | old, outdated equipment, | 1 |
| | (automated | | | | | | unreliable equipment, lack | |
| | staining) | | | | | | of maintenance logs, | |
| | | | | | | | expired service contracts, | |
| | | | | | | | lack of backup device, new | |
| | | | | | | | staff, high turnover in staff | |
| CVS | Coverslipping | Tissue damage | Incorrect | 9 | Human error (Forget slides, | 1 | Poor maintenance, lack of | 1 |
| | (transfer slides to | | Diagnosis | | drop slides, put slide in | | logs, batch processing, high | |
| | CV rack) | | | | incorrect orientation, slides | | volume/low time, etc. | |
| | | | | | dry out due to delay, load | | | |
| | | | | | slide into rack incorrectly, | | | |
| | | | | | forget to obtain slides, etc.) | | | |
| CVS | Coverslipping | Tissue damage | Delay in diagnosis | 3 | Human error (Forget slides, | 1 | Poor maintenance, lack of | 1 |
| | (transfer slides to | | | | drop slides, put slide in | | logs, batch processing, high | |
| | CV rack) | | | | incorrect orientation, slides | | volume/low time, etc. | |
| | | | | | dry out due to delay, load | | | |
| | | | | | slide into rack incorrectly, | | | |
| | | | | | forget to obtain slides, etc.) | | | |
| CVS | Coverslipping | Tissue damage | Rebiopsy | 3 | Human error (Forget slides, | 1 | Poor maintenance, lack of | 1 |
| | (transfer slides to | | | | drop slides, put slide in | | logs, batch processing, high | |
| | CV rack) | | | | incorrect orientation, slides | | volume/low time, etc. | |
| | | | | | dry out due to delay, load | | | |
| | | | | | slide into rack incorrectly, | | | |
| | | | | | forget to obtain slides, etc.) | | | |
| CVS | Coverslipping | Loss of tissue | Incorrect | 9 | Human error (Forget slides, | 1 | Poor maintenance, lack of | 1 |
| | (transfer slides to | | Diagnosis | | drop slides, put slide in | | logs, batch processing, high | |
| | CV rack) | | | | incorrect orientation, slides | | volume/low time, etc. | |
| | | | | | dry out due to delay, load | | | |
| | | | | | slide into rack incorrectly, | | | |
| | | | | | forget to obtain slides, etc.) | | | |
| CVS | Coverslipping | Loss of tissue | Rebiopsy | 3 | Human error (Forget slides, | 1 | Poor maintenance, lack of | 1 |
| | (transfer slides to | | | | drop slides, put slide in | | logs, batch processing, high | |
| | CV rack) | | | | incorrect orientation, slides | | volume/low time, etc. | |
| | | | | | dry out due to delay, load | | | |
| | | | | | slide into rack incorrectly, | | | |
| | | | | | forget to obtain slides, etc.) | | | |
| CVS | Coverslipping | Tissue damage | incorrect diagnosis | 9 | Human error (drop slides | 1 | Batch processing, poor | 1 |
| | (load coverslipper) | | | | inside coverslipper, not seat | | practice, large volume in | |
| | | | | | properly, improper | | limited time, multi-platform | |
| | | | | | positioning, using wrong | | rack/utilization, multiple | |
| | | | | | rack, etc.) | | coverslipping types | |
| CVS | Coverslipping | Tissue damage | rebiopsy | 3 | Human error (drop slides | 1 | Batch processing, poor | 1 |
| | (load coverslipper) | | | | inside coverslipper, not seat | | practice, large volume in | |
| | | | | | properly, improper | | limited time, multi-platform | |
| | | | | | positioning, using wrong | | rack/utilization, multiple | |
| | | | | | rack, etc.) | | coverslipping types | |
| CVS | Coverslipping | Tissue damage | Delay in diagnosis | 3 | Human error (drop slides | 1 | Batch processing, poor | 1 |
| | (load coverslipper) | | | | inside coverslipper, not seat | | practice, large volume in | |
| | | | | | properly, improper | | limited time, multi-platform | |
| | | | | | positioning, using wrong | | rack/utilization, multiple | |
| | | | | | rack, etc.) | | coverslipping types | |
| CVS | Coverslipping | Loss of tissue | incorrect diagnosis | 9 | Human error (drop slides | 1 | Batch processing, poor | 1 |
| | (load coverslipper) | | | | inside coverslipper, not seat | | practice, large volume in | |
| | | | | | properly, improper | | limited time, multi-platform | |
| | | | | | positioning, using wrong | | rack/utilization, multiple | |
| | | | | | rack, etc.) | | coverslipping types | |
| CVS | Coverslipping | Loss of tissue | rebiopsy | 3 | Human error (drop slides | 1 | Batch processing, poor | 1 |
| | (load coverslipper) | | | | inside coverslipper, not seat | | practice, large volume in | |
| | | | | | properly, improper | | limited time, multi-platform | |
| | | | | | positioning, using wrong | | rack/utilization, multiple | |
| | | | | | rack, etc.) | | coverslipping types | |
| CVS | Coverslipping | Loss of tissue | Delay in diagnosis | 3 | Human error (drop slides | 1 | Batch processing, poor | 1 |
| | (load coverslipper) | | | | inside coverslipper, not seat | | practice, large volume in | |
| | | | | | properly, improper | | limited time, multi-platform | |
| | | | | | positioning, using wrong | | rack/utilization, multiple | |
| | | | | | rack, etc.) | | coverslipping types | |
| CVS | Coverslipping | Tissue damage | incorrect diagnosis | 9 | Human error (load wrong | 1 | Multiple coverslippers, | 1 |
| | (consumable | | | | consumable, not load | | multiple slide types, | |
| | check) | | | | consumable, insufficient | | multiple vendors, inventory | |
| | | | | | consumable, dirty | | located far away, new staff, | |
| | | | | | consumable, expired media, | | inexperience | |
| | | | | | etc.) | | | |
| CVS | Coverslipping | Tissue damage | rebiopsy | 3 | Human error (load wrong | 1 | Multiple coverslippers, | 1 |
| | (consumable | | | | consumable, not load | | multiple slide types, | |
| | check) | | | | consumable, insufficient | | multiple vendors, inventory | |
| | | | | | consumable, dirty | | located far away, new staff, | |
| | | | | | consumable, expired media, | | inexperience | |
| | | | | | etc.) | | | |
| CVS | Coverslipping | Tissue damage | Delay in diagnosis | 3 | Human error (load wrong | 3 | Multiple coverslippers, | 1 |
| | (consumable | | | | consumable, not load | | multiple slide types, | |
| | check) | | | | consumable, insufficient | | multiple vendors, inventory | |
| | | | | | consumable, dirty | | located far away, new staff, | |
| | | | | | consumable, expired media, | | inexperience | |
| | | | | | etc.) | | | |
| CVS | Coverslipping | Tissue damage | incorrect diagnosis | 9 | Human error (wrong settings, | 1 | Batch processing, poor | 1 |
| | (instrument check) | | | | lack of priming, speed | | practice, large volume in | |
| | | | | | settings, etc.) | | limited time, multi-platform | |
| | | | | | | | rack/utilization, multiple | |
| | | | | | | | coverslipper types | |
| CVS | Coverslipping | Tissue damage | rebiopsy | 3 | Human error (wrong settings, | 1 | Batch processing, poor | 1 |
| | (instrument check) | | | | lack of priming, speed | | practice, large volume in | |
| | | | | | settings, etc.) | | limited time, multi-platform | |
| | | | | | | | rack/utilization, multiple | |
| | | | | | | | coverslipper types | |
| CVS | Coverslipping | Tissue damage | Delay in diagnosis | 3 | Human error (wrong settings, | 3 | Batch processing, poor | 1 |
| | (instrument check) | | | | lack of priming, speed | | practice, large volume in | |
| | | | | | settings, etc.) | | limited time, multi-platform | |
| | | | | | | | rack/utilization, multiple | |
| | | | | | | | coverslipper types | |
| CVS | Coverslipping | Tissue damage | incorrect diagnosis | 9 | Human error (wrong | 1 | New staff, untrained staff, | 1 |
| | (initiate protocol) | | | | protocol, wrong rack, edit | | poor practice, Batch | |
| | | | | | incorrectly/override, ignore | | processing, poor practice, | |
| | | | | | user actions, etc.) | | large volume in limited | |
| | | | | | | | time, multi-platform rack/ | |
| | | | | | | | utilization, multiple | |
| | | | | | | | coverslipper types | |
| CVS | Coverslipping | Tissue damage | rebiopsy | 3 | Human error (wrong | 1 | New staff, untrained staff, | 1 |
| | (initiate protocol) | | | | protocol, wrong rack, edit | | poor practice, Batch | |
| | | | | | incorrectly/override, ignore | | processing, poor practice, | |
| | | | | | user actions, etc.) | | large volume in limited | |
| | | | | | | | time, multi-platform rack/ | |
| | | | | | | | utilization, multiple | |
| | | | | | | | coverslipper types | |
| CVS | Coverslipping | Tissue damage | Delay in diagnosis | 3 | Human error (wrong | 1 | New staff, untrained staff, | 1 |
| | (initiate protocol) | | | | protocol, wrong rack, edit | | poor practice, Batch | |
| | | | | | incorrectly/override, ignore | | processing, poor practice, | |
| | | | | | user actions, etc.) | | large volume in limited | |
| | | | | | | | time, multi-platform rack/ | |
| | | | | | | | utilization, multiple | |
| | | | | | | | coverslipper types | |
| CVS | Coverslipping | Tissue damage | incorrect diagnosis | 9 | Device malfunction | 1 | old, outdated equipment, | 1 |
| | (initiate protocol) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| CVS | Coverslipping | Tissue damage | rebiopsy | 3 | Device malfunction | 1 | old, outdated equipment, | 1 |
| | (initiate protocol) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| CVS | Coverslipping | Tissue damage | Delay in diagnosis | 3 | Device malfunction | 9 | old, outdated equipment, | 1 |
| | (initiate protocol) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| CVS | Coverslipping | Processor won't | Delay in diagnosis | 3 | Human error (forgot to hit | 1 | new personnel, lab | 1 |
| | (initiate protocol) | initiate (as result | | | ″start″, forgot to close door, | | cleanliness, dirty | |
| | | of human error) | | | etc.) | | instrument | |
| CVS | Coverslipping | Processor won't | Delay in diagnosis | 3 | Device malfunction | 9 | old, outdated equipment, | 1 |
| | (initiate protocol) | initiate | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| CVS | Coverslipping | Tissue damage | incorrect diagnosis | 9 | Device malfunction | 1 | old, outdated equipment, | 1 |
| | (automated | | | | | | unreliable equipment, lack | |
| | coverslipping) | | | | | | of maintenance logs, | |
| | | | | | | | expired service contracts, | |
| | | | | | | | lack of backup device, new | |
| | | | | | | | staff, high turnover in staff | |
| CVS | Coverslipping | Tissue damage | rebiopsy | 3 | Device malfunction | 1 | old, outdated equipment, | 1 |
| | (automated | | | | | | unreliable equipment, lack | |
| | coverslipping) | | | | | | of maintenance logs, | |
| | | | | | | | expired service contracts, | |
| | | | | | | | lack of backup device, new | |
| | | | | | | | staff, high turnover in staff | |
| CVS | Coverslipping | Tissue damage | Delay in diagnosis | 3 | Device malfunction | 9 | old, outdated equipment, | 1 |
| | (automated | | | | | | unreliable equipment, lack | |
| | coverslipping) | | | | | | of maintenance logs, | |
| | | | | | | | expired service contracts, | |
| | | | | | | | lack of backup device, new | |
| | | | | | | | staff, high turnover in staff | |
| CVS | Coverslipping | Loss of tissue | incorrect diagnosis | 9 | Device malfunction | 1 | old, outdated equipment, | 1 |
| | (automated | | | | | | unreliable equipment, lack | |
| | coverslipping) | | | | | | of maintenance logs, | |
| | | | | | | | expired service contracts, | |
| | | | | | | | lack of backup device, new | |
| | | | | | | | staff, high turnover in staff | |
| CVS | Coverslipping | Loss of tissue | rebiopsy | 3 | Device malfunction | 1 | old, outdated equipment, | 1 |
| | (automated | | | | | | unreliable equipment, lack | |
| | coverslipping) | | | | | | of maintenance logs, | |
| | | | | | | | expired service contracts, | |
| | | | | | | | lack of backup device, new | |
| | | | | | | | staff, high turnover in staff | |
| CVS | Coverslipping | Loss of tissue | Delay in diagnosis | 3 | Device malfunction | 3 | old, outdated equipment, | 1 |
| | (automated | | | | | | unreliable equipment, lack | |
| | coverslipping) | | | | | | of maintenance logs, | |
| | | | | | | | expired service contracts, | |
| | | | | | | | lack of backup device, new | |
| | | | | | | | staff, high turnover in staff | |
| CVS | Coverslipping | tissue damage | incorrect diagnosis | 9 | Human error (too short, skip | 1 | Lack of timers, poor | 1 |
| | (Dry slides) | | | | step, move coverslip, etc.) | | maintenance, lack of logs, | |
| | | | | | | | batch processing, high | |
| | | | | | | | volume/low time, etc. | |
| CVS | Coverslipping | tissue damage | rebiopsy | 3 | Human error (too short, skip | 1 | Lack of timers, poor | 1 |
| | (Dry slides) | | | | step, move coverslip, etc.) | | maintenance, lack of logs, | |
| | | | | | | | batch processing, high | |
| | | | | | | | volume/low time, etc. | |
| CVS | Coverslipping | tissue damage | Delay in diagnosis | 3 | Human error (too short, skip | 1 | Lack of timers, poor | 1 |
| | (Dry slides) | | | | step, move coverslip, etc.) | | maintenance, lack of logs, | |
| | | | | | | | batch processing, high | |
| | | | | | | | volume/low time, etc. | |
| CVS | Coverslipping | Loss of tissue | incorrect diagnosis | 9 | Human error (too short, skip | 1 | Lack of timers, poor | 1 |
| | (Dry slides) | | | | step, move coverslip, etc.) | | maintenance, lack of logs, | |
| | | | | | | | batch processing, high | |
| | | | | | | | volume/low time, etc. | |
| CVS | Coverslipping | Loss of tissue | rebiopsy | 3 | Human error (too short, skip | 1 | Lack of timers, poor | 1 |
| | (Dry slides) | | | | step, move coverslip, etc.) | | maintenance, lack of logs, | |
| | | | | | | | batch processing, high | |
| | | | | | | | volume/low time, etc. | |
| CVS | Coverslipping | Loss of tissue | Delay in diagnosis | 3 | Human error (too short, skip | 1 | Lack of timers, poor | 1 |
| | (Dry slides) | | | | step, move coverslip, etc.) | | maintenance, lack of logs, | |
| | | | | | | | batch processing, high | |
| | | | | | | | volume/low time, etc. | |
| Prep | Case Assembly | patient/data | incorrect diagnosis | 9 | Human error (transposed, lost | 3 | Stacks of paper, stacks of | 1 |
| | (slides out of rack/ | mismatch | | | paperwork, mismatch, lost | | slides, manual matching, | |
| | match to | | | | label, smudged label, etc.) | | lack of specimen tracking, | |
| | paperwork/placed | | | | | | lack of space, etc. | |
| | into slide tray) | | | | | | | |
| Prep | Case Assembly | patient/data | rebiopsy | 3 | Human error (transposed, lost | 1 | Stacks of paper, stacks of | 1 |
| | (slides out of rack/ | mismatch | | | paperwork, mismatch, lost | | slides, manual matching, | |
| | match to | | | | label, smudged label, etc.) | | lack of specimen tracking, | |
| | paperwork/placed | | | | | | lack of space, etc. | |
| | into slide tray) | | | | | | | |
| Prep | Case Assembly | patient/data | Delay in diagnosis | 3 | Human error (transposed, lost | 9 | Stacks of paper, stacks of | 1 |
| | (slides out of rack/ | mismatch | | | paperwork, mismatch, lost | | slides, manual matching, | |
| | match to | | | | label, smudged label, etc.) | | lack of specimen tracking, | |
| | paperwork/placed | | | | | | lack of space, etc. | |
| | into slide tray) | | | | | | | |
| Prep | Case Assembly | patient/data | incorrect diagnosis | 9 | Human error (wrong label, | 3 | Stacks of paper, stacks of | 1 |
| | (label - if | mismatch | | | wrong slide, wrong block | | slides, manual matching, | |
| | necessary) | | | | identifier on wrong slide - but | | lack of specimen tracking, | |
| | | | | | right patient, etc.) | | lack of space, etc. | |
| Prep | Case Assembly | patient/data | rebiopsy | 3 | Human error (wrong label, | 1 | Stacks of paper, stacks of | 1 |
| | (label - if | mismatch | | | wrong slide, wrong block | | slides, manual matching, | |
| | necessary) | | | | identifier on wrong slide - but | | lack of specimen tracking, | |
| | | | | | right patient, etc.) | | lack of space, etc. | |
| Prep | Case Assembly | patient/data | Delay in diagnosis | 3 | Human error (wrong label, | 9 | Stacks of paper, stacks of | 1 |
| | (label - if | mismatch | | | wrong slide, wrong block | | slides, manual matching, | |
| | necessary) | | | | identifier on wrong slide - but | | lack of specimen tracking, | |
| | | | | | right patient, etc.) | | lack of space, etc. | |
| Prep | Case Assembly | physician/ | Delay in diagnosis | 3 | Human error (assign incorrect | 3 | Large volume of | 1 |
| | (sort to | patient/data | | | pathologist, transpose slides/ | | pathologists, lack of digital | |
| | pathologist) | mismatch | | | paperwork, etc.) | | pathology, Stacks of paper, | |
| | | | | | | | stacks of slides, manual | |
| | | | | | | | matching, lack of specimen | |
| | | | | | | | tracking, lack of space, etc. | |
| IHC | IHC (Unloading/ | Tissue damage | Delay in diagnosis | 3 | Human error (Forget tissue, | 1 | Lack of specimen tracking, | 1 |
| | Transport to | | | | drop tissue, forget to obtain | | batch processing, messy | |
| | microtomy) | | | | tissue, etc.) | | workstation, lack of visual | |
| | | | | | | | cues, lack of identified | |
| | | | | | | | tissue cassettes | |
| IHC | IHC (Unloading/ | Loss of tissue | Incorrect | 9 | Human error (Forget tissue, | 1 | Lack of specimen tracking, | 1 |
| | Transport to | | Diagnosis | | drop tissue, forget to obtain | | batch processing, messy | |
| | microtomy) | | | | tissue, etc.) | | workstation, lack of visual | |
| | | | | | | | cues, lack of identified | |
| | | | | | | | tissue cassettes | |
| IHC | IHC (Microtomy | Tissue damage | incorrect diagnosis | 9 | Human error (left cassette on | 1 | Batch processing, high | 1 |
| | (ice)) | | | | ice too long, left cassette on | | volumes, full ice trays (with | |
| | | | | | ice too short, etc.) | | cassettes), melting ice | |
| | | | | | | | blocks, processing artifacts, | |
| | | | | | | | sunken blocks after storage | |
| IHC | IHC (Microtomy | Tissue damage | Delay in diagnosis | 3 | Human error (left cassette on | 1 | Batch processing, high | 1 |
| | (ice)) | | | | ice too long, left cassette on | | volumes, full ice trays (with | |
| | | | | | ice too short, etc.) | | cassettes), melting ice | |
| | | | | | | | blocks, processing artifacts, | |
| | | | | | | | sunken blocks after storage | |
| IHC | IHC (Microtomy | Loss of tissue | incorrect diagnosis | 9 | Human error (left cassette on | 1 | Batch processing, high | 1 |
| | (ice)) | | | | ice too long, left cassette on | | volumes, full ice trays (with | |
| | | | | | ice too short, etc.) | | cassettes), melting ice | |
| | | | | | | | blocks, processing artifacts, | |
| | | | | | | | sunken blocks after storage | |
| IHC | IHC (Microtomy | Loss of tissue | Delay in diagnosis | 3 | Human error (left cassette on | 1 | Batch processing, high | 1 |
| | (ice)) | | | | ice too long, left cassette on | | volumes, full ice trays (with | |
| | | | | | ice too short, etc.) | | cassettes), melting ice | |
| | | | | | | | blocks, processing artifacts, | |
| | | | | | | | sunken blocks after storage | |
| IHC | IHC (Microtomy | Patient/data | incorrect diagnosis | 9 | Human error (transpose, | 1 | batch processing, multiple | 1 |
| | (matching, printing | mismatch | | | wrong side, wrong block, | | consumables out at once, | |
| | slide, finding slide, | | | | omit slide, omit block, | | messy workspace, high | |
| | etc.)) | | | | selected incorrect test panel, | | volume, lack of specimen | |
| | | | | | incorrect #slides pulled, etc.) | | tracking | |
| IHC | IHC (Microtomy | Patient/data | Delay in diagnosis | 3 | Human error (transpose, | 3 | batch processing, multiple | 1 |
| | (matching, printing | mismatch | | | wrong side, wrong block, | | consumables out at once, | |
| | slide, finding slide, | | | | omit slide, omit block, | | messy workspace, high | |
| | etc.)) | | | | selected incorrect test panel, | | volume, lack of specimen | |
| | | | | | incorrect #slides pulled, etc.) | | tracking | |
| IHC | IHC (Microtomy | Tissue damage | incorrect diagnosis | 9 | Device malfunction | 1 | old, outdated equipment, | 1 |
| | (cutting)) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| IHC | IHC (Microtomy | Tissue damage | Delay in diagnosis | 3 | Device malfunction | 1 | old, outdated equipment, | 1 |
| | (cutting)) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| IHC | IHC (Microtomy | Loss of tissue | incorrect diagnosis | 9 | Device malfunction | 1 | old, outdated equipment, | 1 |
| | (cutting)) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| IHC | IHC (Microtomy | Loss of tissue | Delay in diagnosis | 3 | Device malfunction | 1 | old, outdated equipment, | 1 |
| | (cutting)) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| IHC | IHC (Microtomy | Tissue damage | incorrect diagnosis | 9 | Human error (too hot, too | 1 | dirty bath, lack of | 1 |
| | (prepare and using | | | | cold, not enough H2O, too | | thermometer, lack of | |
| | water bath)) | | | | much H2O, contaminated | | temperature gauge, | |
| | | | | | water, in too long, not long | | additives out next to | |
| | | | | | enough, additives, etc.) | | waterbath, high volume, | |
| | | | | | | | batch processing | |
| IHC | IHC (Microtomy | Tissue damage | Delay in diagnosis | 3 | Human error (too hot, too | 3 | dirty bath, lack of | 1 |
| | (prepare and using | | | | cold, not enough H2O, too | | thermometer, lack of | |
| | water bath)) | | | | much H2O, contaminated | | temperature gauge, | |
| | | | | | water, in too long, not long | | additives out next to | |
| | | | | | enough, additives, etc.) | | waterbath, high volume, | |
| | | | | | | | batch processing | |
| IHC | IHC (Microtomy | Loss of tissue | incorrect diagnosis | 9 | Human error (too hot, too | 1 | dirty bath, lack of | 1 |
| | (prepare and using | | | | cold, not enough H2O, too | | thermometer, lack of | |
| | water bath)) | | | | much H2O, contaminated | | temperature gauge, | |
| | | | | | water, in too long, not long | | additives out next to | |
| | | | | | enough, additives, etc.) | | waterbath, high volume, | |
| | | | | | | | batch processing | |
| IHC | IHC (Microtomy | Loss of tissue | Delay in diagnosis | 3 | Human error (too hot, too | 3 | dirty bath, lack of | 1 |
| | (prepare and using | | | | cold, not enough H2O, too | | thermometer, lack of | |
| | water bath)) | | | | much H2O, contaminated | | temperature gauge, | |
| | | | | | water, in too long, not long | | additives out next to | |
| | | | | | enough, additives, etc.) | | waterbath, high volume, | |
| | | | | | | | batch processing | |
| IHC | IHC (Microtomy | Tissue damage | incorrect diagnosis | 9 | Device malfunction | 1 | old, outdated equipment, | 1 |
| | (prepare and using | | | | | | unreliable equipment, lack | |
| | water bath)) | | | | | | of maintenance logs, | |
| | | | | | | | expired service contracts, | |
| | | | | | | | lack of backup device, new | |
| | | | | | | | staff, high turnover in staff | |
| IHC | IHC (Microtomy | Tissue damage | Delay in diagnosis | 3 | Device malfunction | 1 | old, outdated equipment, | 1 |
| | (prepare and using | | | | | | unreliable equipment, lack | |
| | water bath)) | | | | | | of maintenance logs, | |
| | | | | | | | expired service contracts, | |
| | | | | | | | lack of backup device, new | |
| | | | | | | | staff, high turnover in staff | |
| IHC | IHC (Microtomy | Loss of tissue | incorrect diagnosis | 9 | Device malfunction | 1 | old, outdated equipment, | 1 |
| | (prepare and using | | | | | | unreliable equipment, lack | |
| | water bath)) | | | | | | of maintenance logs, | |
| | | | | | | | expired service contracts, | |
| | | | | | | | lack of backup device, new | |
| | | | | | | | staff, high turnover in staff | |
| IHC | IHC (Microtomy | Loss of tissue | Delay in diagnosis | 3 | Device malfunction | 1 | old, outdated equipment, | 1 |
| | (prepare and using | | | | | | unreliable equipment, lack | |
| | water bath)) | | | | | | of maintenance logs, | |
| | | | | | | | expired service contracts, | |
| | | | | | | | lack of backup device, new | |
| | | | | | | | staff, high turnover in staff | |
| IHC | IHC (Microtomy | Tissue damage | incorrect diagnosis | 9 | Human error (orientation, | 1 | new staff, high volume, | 1 |
| | (transfer from | | | | incorrect selection, add | | waterbath is full, dirty | |
| | water bath to slide | | | | artifact, contaminate, dirty | | water, batch processing, | |
| | and into slide | | | | slides, incorrect consumables, | | additives sitting out, poor | |
| | rack)) | | | | incomplete drying (i.e. flick | | processing, artifact in final | |
| | | | | | water off), etc.) | | slides | |
| IHC | IHC (Microtomy | Tissue damage | Delay in diagnosis | 3 | Human error (orientation, | 1 | new staff, high volume, | 1 |
| | (transfer from | | | | incorrect selection, add | | waterbath is full, dirty | |
| | water bath to slide | | | | artifact, contaminate, dirty | | water, batch processing, | |
| | and into slide | | | | slides, incorrect consumables, | | additives sitting out, poor | |
| | rack)) | | | | incomplete drying (i.e. flick | | processing, artifact in final | |
| | | | | | water off), etc.) | | slides | |
| IHC | IHC (Microtomy | Loss of tissue | incorrect diagnosis | 9 | Human error (orientation, | 1 | new staff, high volume, | 1 |
| | (transfer from | | | | incorrect selection, add | | waterbath is full, dirty | |
| | water bath to slide | | | | artifact, contaminate, dirty | | water, batch processing, | |
| | and into slide | | | | slides, incorrect consumables, | | additives sitting out, poor | |
| | rack)) | | | | incomplete drying (i.e. flick | | processing, artifact in final | |
| | | | | | water off), etc.) | | slides | |
| IHC | IHC (Microtomy | Loss of tissue | Delay in diagnosis | 3 | Human error (orientation, | 3 | new staff, high volume, | 1 |
| | (transfer from | | | | incorrect selection, add | | waterbath is full, dirty | |
| | water bath to slide | | | | artifact, contaminate, dirty | | water, batch processing, | |
| | and into slide | | | | slides, incorrect consumables, | | additives sitting out, poor | |
| | rack)) | | | | incomplete drying (i.e. flick | | processing, artifact in final | |
| | | | | | water off), etc.) | | slides | |
| IHC | IHC (Microtomy | patient/data | incorrect diagnosis | 9 | Human error (tissue placed | 1 | batch processing, high | 1 |
| | (transfer from | mismatch | | | onto wrong slide) | | volumes, lack of specimen | |
| | water bath to slide | | | | | | tracking, batch slides | |
| | and into slide | | | | | | printed out, stacks of slides, | |
| | rack)) | | | | | | handwritten slides, etc. | |
| IHC | IHC (Microtomy | patient/data | Delay in diagnosis | 3 | Human error (tissue placed | 3 | batch processing, high | 1 |
| | (transfer from | mismatch | | | onto wrong slide) | | volumes, lack of specimen | |
| | water bath to slide | | | | | | tracking, batch slides | |
| | and into slide | | | | | | printed out, stacks of slides, | |
| | rack)) | | | | | | handwritten slides, etc. | |
| IHC | IHC (Dry slides) | tissue damage | incorrect diagnosis | 9 | Human error (too long, too | 3 | Lack of thermometers, | 1 |
| | | | | | short, skip step, too hot, too | | timers, poor maintenance, | |
| | | | | | cold, etc.) | | lack of logs, batch | |
| | | | | | | | processing, high volume/ | |
| | | | | | | | low time, etc. | |
| IHC | IHC (Dry slides) | tissue damage | Delay in diagnosis | 3 | Human error (too long, too | 3 | Lack of thermometers, | 1 |
| | | | | | short, skip step, too hot, too | | timers, poor maintenance, | |
| | | | | | cold, etc.) | | lack of logs, batch | |
| | | | | | | | processing, high volume/ | |
| | | | | | | | low time, etc. | |
| IHC | IHC (label slides) | patient/data | incorrect diagnosis | 9 | Human error (labeling error, | 1 | stacks of labels, stacks of | 1 |
| | | mismatch | | | transpose, forget to label, flag | | slides, batch processing, | |
| | | | | | label, smudge label, wrong | | high volume testing | |
| | | | | | area of slide, etc.) | | | |
| IHC | IHC (label slides) | patient/data | Delay in diagnosis | 3 | Human error (labeling error, | 3 | stacks of labels, stacks of | 1 |
| | | mismatch | | | transpose, forget to label, flag | | slides, batch processing, | |
| | | | | | label, smudge label, wrong | | high volume testing | |
| | | | | | area of slide, etc.) | | | |
| IHC | IHC (initiate | Tissue damage | incorrect diagnosis | 9 | Human error (wrong | 1 | Batch processing, poor | 1 |
| | protocol) | | | | protocol, edit incorrectly/ | | practice, large volume in | |
| | | | | | override, override warnings, | | limited time, multi-platform | |
| | | | | | ignore user actions, etc.) | | rack/utilization, multiple, | |
| | | | | | | | stainer types | |
| IHC | IHC (initiate | Tissue damage | Delay in diagnosis | 3 | Human error (wrong | 3 | Batch processing, poor | 1 |
| | protocol) | | | | protocol, edit incorrectly/ | | practice, large volume in | |
| | | | | | override, override warnings, | | limited time, multi-platform | |
| | | | | | ignore user actions, etc.) | | rack/utilization, multiple, | |
| | | | | | | | stainer types | |
| IHC | IHC (initiate | Tissue damage | incorrect diagnosis | 9 | Device malfunction | 1 | old, outdated equipment, | 1 |
| | protocol) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| IHC | IHC (initiate | Tissue damage | Delay in diagnosis | 3 | Device malfunction | 1 | old, outdated equipment, | 1 |
| | protocol) | | | | | | unreliable equipment, lack | |
| | | | | | | | of maintenance logs, | |
| | | | | | | | expired service contracts, | |
| | | | | | | | lack of backup device, new | |
| | | | | | | | staff, high turnover in staff | |
| IHC | IHC (initiate | Stainer won't | Delay in diagnosis | 3 | Human error (forgot to hit | 1 | Batch processing, poor | 1 |
| | protocol) | initiate (as result | | | ″start″, forgot to close door, | | practice, large volume in, | |
| | | of human error) | | | keep lids on, etc.) | | limited time, multi-platform | |
| | | | | | | | rack/utilization, multiple | |
| | | | | | | | stainer types | |
| IHC | IHC (initiate | Stainer won't | Delay in diagnosis | 3 | Device malfunction | 1 | old, outdated equipment, | 1 |
| | protocol) | initiate | | | | | unreliable equipment, lack | |
| | | | | | | | of maintenance logs, | |
| | | | | | | | expired service contracts, | |
| | | | | | | | lack of back device, new | |
| | | | | | | | staff, high turnover in staff | |
| IHC | IHC (automated | Tissue damage | incorrect diagnosis | 9 | Device malfunction | 1 | old, outdated equipment, | 1 |
| | staining) | | | | | | unreliable equipment, lack | |
| | | | | | | | of maintenance logs, | |
| | | | | | | | expired service contracts, | |
| | | | | | | | lack of back device, new | |
| | | | | | | | staff, high turnover in staff | |
| IHC | IHC (automated | Tissue damage | Delay in diagnosis | 3 | Device malfunction | 1 | old, outdated equipment, | 1 |
| | staining) | | | | | | unreliable equipment, lack | |
| | | | | | | | of maintenance logs, | |
| | | | | | | | expired service contracts, | |
| | | | | | | | lack of back device, new | |
| | | | | | | | staff, high turnover in staff | |
| IHC | IHC (automated | Loss of tissue | incorrect diagnosis | 9 | Device malfunction | 1 | old, outdated equipment, | 1 |
| | staining) | | | | | | unreliable equipment, lack | |
| | | | | | | | of maintenance logs, | |
| | | | | | | | expired service contracts, | |
| | | | | | | | lack of back device, new | |
| | | | | | | | staff, high turnover in staff | |
| IHC | IHC (automated | Loss of tissue | Delay in diagnosis | 3 | Device malfunction | 1 | old, outdated equipment, | 1 |
| | staining) | | | | | | unreliable equipment, lack | |
| | | | | | | | of maintenance logs, | |
| | | | | | | | expired service contracts, | |
| | | | | | | | lack of back device, new | |
| | | | | | | | staff, high turnover in staff | |
| IHC | IHC | Tissue damage | incorrect diagnosis | 9 | Human error (drop slides | 1 | Batch processing, poor | 1 |
| | (Coverslipping | | | | inside coverslipper, not seat | | practice, large volume in | |
| | (load | | | | properly, improper | | limited time, multi-platform | |
| | coverslipper)) | | | | positioning, using wrong | | rack/utilization, multiple | |
| | | | | | rack, etc.) | | coverslipping types | |
| IHC | IHC | Tissue damage | Delay in diagnosis | 3 | Human error (drop slides | 1 | Batch processing, poor | 1 |
| | (Coverslipping | | | | inside coverslipper, not seat | | practice, large volume in | |
| | (load | | | | properly, improper | | limited time, multi-platform | |
| | coverslipper)) | | | | positioning, using wrong | | rack/utilization, multiple | |
| | | | | | rack, etc.) | | coverslipping types | |
| IHC | IHC | Loss of tissue | incorrect diagnosis | 9 | Human error (drop slides | 1 | Batch processing, poor | 1 |
| | (Coverslipping | | | | inside coverslipper, not seat | | practice, large volume in | |
| | (load | | | | properly, improper | | limited time, multi-platform | |
| | coverslipper)) | | | | positioning, using wrong | | rack/utilization, multiple | |
| | | | | | rack, etc.) | | coverslipping types | |
| IHC | IHC | Loss of tissue | Delay in diagnosis | 3 | Human error (drop slides | 1 | Batch processing, poor | 1 |
| | (Coverslipping | | | | inside coverslipper, not seat | | practice, large volume in | |
| | (load | | | | properly, improper | | limited time, multi-platform | |
| | coverslipper)) | | | | positioning, using wrong | | rack/utilization, multiple | |
| | | | | | rack, etc.) | | coverslipping types | |
| IHC | IHC | Tissue damage | incorrect diagnosis | 9 | Human error (wrong settings, | 1 | Batch processing, poor | 1 |
| | (Coverslipping | | | | lack of priming, speed | | practice, large volume in | |
| | (instrument | | | | settings, etc.) | | limited time, multi-platform | |
| | check)) | | | | | | rack/utilization, multiple | |
| | | | | | | | coverslipper types | |
| IHC | IHC | Tissue damage | Delay in diagnosis | 3 | Human error (wrong settings, | 3 | Batch processing, poor | 1 |
| | (Coverslipping | | | | lack of priming, speed | | practice, large volume in | |
| | (instrument | | | | settings, etc.) | | limited time, multi-platform | |
| | check)) | | | | | | rack/utilization, multiple | |
| | | | | | | | coverslipper types | |
| IHC | IHC | Tissue damage | incorrect diagnosis | 9 | Human error (wrong | 1 | New staff, untrained staff, | 1 |
| | (Coverslipping | | | | protocol, wrong rack, edit | | poor practice, Batch | |
| | (initiate protocol)) | | | | incorrectly/override, ignore | | processing, poor practice, | |
| | | | | | user actions, etc.) | | large volume in limited | |
| | | | | | | | time, multi-platform rack/ | |
| | | | | | | | utilization, multiple | |
| | | | | | | | coverslipper types | |
| IHC | IHC | Tissue damage | Delay in diagnosis | 3 | Human error (wrong | 1 | New staff, untrained staff, | 1 |
| | (Coverslipping | | | | protocol, wrong rack, edit | | poor practice, Batch | |
| | (initiate protocol)) | | | | incorrectly/override, ignore | | processing, poor practice, | |
| | | | | | user actions, etc.) | | large volume in limited | |
| | | | | | | | time, multi-platform rack/ | |
| | | | | | | | utilization, multiple | |
| | | | | | | | coverslipper types | |
| IHC | IHC | Tissue damage | incorrect diagnosis | 9 | Device malfunction | 1 | old, outdated equpiment, | 1 |
| | (Coverslipping | | | | | | unreliable equipment, lack | |
| | (initiate protocol) | | | | | | of maintenance logs, | |
| | | | | | | | expired service contracts, | |
| | | | | | | | lack of backup device, new | |
| | | | | | | | staff, high turnover in staff | |
| IHC | IHC | Tissue damage | Delay in diagnosis | 3 | Device malfunction | 9 | old, outdated equpiment, | 1 |
| | (Coverslipping | | | | | | unreliable equipment, lack | |
| | (initiate protocol) | | | | | | of maintenance logs, | |
| | | | | | | | expired service contracts, | |
| | | | | | | | lack of backup device, new | |
| | | | | | | | staff, high turnover in staff | |
| IHC | IHC | Processor won't | Delay in diagnosis | 3 | Device malfunction | 9 | old, outdated equipment, | 1 |
| | (Coverslipping | initiate | | | | | unreliable equipment, lack | |
| | (initiate protocol)) | | | | | | of maintenance logs, | |
| | | | | | | | expired service contracts, | |
| | | | | | | | lack of backup device, new | |
| | | | | | | | staff, high turnover in staff | |
| IHC | IHC | Tissue damage | incorrect diagnosis | 9 | Device malfunction | 1 | old, outdated equipment, | 1 |
| | (Coverslipping | | | | | | unreliable equipment, lack | |
| | (automated | | | | | | of maintenance logs, | |
| | coverslipping)) | | | | | | expired service contracts, | |
| | | | | | | | lack of backup device, new | |
| | | | | | | | staff, high turnover in staff | |
| IHC | IHC | Tissue damage | Delay in diagnosis | 3 | Device malfunction | 9 | old, outdated equipment, | 1 |
| | (Coverslipping | | | | | | unreliable equipment, lack | |
| | (automated | | | | | | of maintenance logs, | |
| | coverslipping)) | | | | | | expired service contracts, | |
| | | | | | | | lack of backup device, new | |
| | | | | | | | staff, high turnover in staff | |
| IHC | IHC | Loss of tissue | incorrect diagnosis | 9 | Device malfunction | 1 | old, outdated equipment, | 1 |
| | (Coverslipping | | | | | | unreliable equipment, lack | |
| | (automated | | | | | | of maintenance logs, | |
| | coverslipping)) | | | | | | expired service contracts, | |
| | | | | | | | lack of backup device, new | |
| | | | | | | | staff, high turnover in staff | |
| IHC | IHC | Loss of tissue | Delay in diagnosis | 3 | Device malfunction | 3 | old, outdated equipment, | 1 |
| | (Coverslipping | | | | | | unreliable equipment, lack | |
| | (automated | | | | | | of maintenance logs, | |
| | coverslipping)) | | | | | | expired service contracts, | |
| | | | | | | | lack of backup device, new | |
| | | | | | | | staff, high turnover in staff | |
| IHC | IHC | tissue damage | incorrect diagnosis | 9 | Human error (too short, skip | 1 | Lack of timers, poor | 1 |
| | (Coverslipping | | | | step, move coverslip, etc.) | | maintenance, lack of logs, | |
| | (Dry slides)) | | | | | | batch processing, high | |
| | | | | | | | volume/low time, etc. | |
| IHC | IHC | tissue damage | Delay in diagnosis | 3 | Human error (too short, skip | 1 | Lack of timers, poor | 1 |
| | (Coverslipping | | | | step, move coverslip, etc.) | | maintenance, lack of logs, | |
| | (Dry slides)) | | | | | | batch processing, high | |
| | | | | | | | volume/low time, etc. | |
| IHC | IHC | Loss of tissue | incorrect diagnosis | 9 | Human error (too short, skip | 1 | Lack of timers, poor | 1 |
| | (Coverslipping | | | | step, move coverslip, etc.) | | maintenance, lack of logs, | |
| | (Dry slides)) | | | | | | batch processing, high | |
| | | | | | | | volume/low time, etc. | |
| IHC | IHC | Loss of tissue | Delay in diagnosis | 3 | Human error (too short, skip | 1 | Lack of timers, poor | 1 |
| | (Coverslipping | | | | step, move coverslip, etc.) | | maintenance, lack of logs, | |
| | (Dry slides)) | | | | | | batch processing, high | |
| | | | | | | | volume/low time, etc. | |

VI. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An audit device comprising a processor configured to execute instructions stored on a memory, a display operable by the processor to provide information to a user, and a user input device operable by the user to provide input to the processor, wherein the processor is configured to: provide a set of assessment modules via the display, wherein each of the set of assessment modules describes one or more observable risk indicators of an audit site, receive a set of responses, wherein each response is associated with an assessment module of the set of assessment module and indicates whether an observable risk indicator is present at the audit site; determine a set of risks based upon the set of responses, wherein each risk of the set of risks corresponds with at least one observable risk indicator within the set of responses; calculate a total risk score for the audit site based upon the set of risks; and display the total risk score.

### Example 2

The audit device of Example 1, wherein the set of risks is determined from a risk database including at least seven hundred risks, and wherein each risk in the set of risks includes a severity score and an occurrence score, and wherein the processor is configured to calculate the total risk score based upon the severity score and the occurrence score of each risk.

### Example 3

The audit device of Example 2, wherein each risk of the set of risks is associated with a capability of the audit site, and wherein the processor is configured to calculate and display a capability total risk score for each capability based upon the set of risks.

### Example 4

The audit device of Example 3, wherein each capability is within a set of capabilities including radiology, transfer to pathology, grossing, fixation, embedding, monotomy, staining, coverslipping, slide preparation, and immunohistochemistry.

### Example 5

The audit device of any one or more of Examples 3 through 4, wherein the processor is further configured to display the set of capability total risk scores sorted from highest to lowest.

### Example 6

The audit device of any one or more of Examples 1 through 5, further comprising an image capture device, wherein the processor is further configured to: determine that an assessment module of the set of assessment modules is configured to request an observable risk indicator image; when providing the assessment module via the display, provide an image capture interface operable to capture an image of that observable risk indicator with the image capture device; and associate the image with a response for the assessment module.

### Example 7

The audit device of any one or more of Examples 1 through 6, further comprising a step counting device, wherein the processor is further configured to: determine that an assessment module of the set of assessment modules is configured to request a distance measurement; when providing the assessment module via the display, provide a distance measurement interface operable to measure a distance between a start location and a destination location using the step counting device, and associate the distance with a response for the assessment module.

### Example 8

The audit device of any one or more of Examples 1 through 7, wherein the processor is further configured to, when providing the set of assessment modules: determine a location of the audit device; determine an assessment module of the set of assessment modules that is associated with the location; and provide the assessment module.

### Example 9

The audit device of Example 8, further comprising a global positioning device, wherein the processor is further configured to determine the location using the global positioning device.

### Example 10

The audit device of any one or more of Examples 1 through 9, wherein the processor is further configured to: associate a post-control risk score with each risk of the set of risks based on a severity score associated with each risk and a post-control occurrence score associated with each risk; calculate a total post-occurrence risk score for the audit site based upon the set of risks and the post-control risk score for each risk; and display the total post-occurrence risk score.

### Example 11

A method for providing a guided assessment of risk at an audit site comprising: providing a set of assessment modules via a display of an audit device, wherein each of the set of assessment modules describes one or more observable risk indicators of an audit site; receiving a set of responses via a user input device of the audit device, wherein each response is associated with an assessment module of the set of assessment module and indicates whether an observable risk indicator is present at the audit site; determining a set of risks based upon the set of responses, wherein each risk of the set of risks corresponds with at least one observable risk indicator within the set of responses; calculating a total risk score for the audit site based upon the set of risks; and displaying the total risk score.

### Example 12

The method of Example 11, wherein the set of risks is determined from a risk database including at least seven hundred risks, and wherein each risk in the set of risks includes a severity score and an occurrence score, the method further comprising calculating the total risk score based upon the severity score and the occurrence score of each risk.

### Example 13

The method of Example 12, wherein each risk of the set of risks is associated with a capability of the audit site, the method further comprising calculating and displaying a capability total risk score for each capability based upon the set of risks.

### Example 14

The method of Example 13, wherein each capability is within a set of capabilities including radiology, transfer to pathology, grossing, fixation, embedding, monotomy, staining, coverslipping, slide preparation, and immunohistochemistry.

### Example 15

The method of any one or more of Examples 13 through 14, further comprising displaying the set of capability total risk scores sorted from highest to lowest.

### Example 16

The method of any one or more of Examples 11 through 15, further comprising: determining that an assessment module of the set of assessment modules is configured to request an observable risk indicator image: when providing the assessment module via the display, providing an image capture interface operable to capture an image of that observable risk indicator with an image capture device of the audit device; and associating the image with a response for the assessment module.

### Example 17

The method of any one or more of Examples 11 through 16, further comprising: determining that an assessment module of the set of assessment modules is configured to request a distance measurement; when providing the assessment module via the display, providing a distance measurement interface operable to measure a distance between a start location and a destination location using a step counting device of the audit device; and associating the distance with a response for the assessment module.

### Example 18

The method of any one or more of Examples 11 through 17, further comprising, when providing the set of assessment modules: determining a location of the audit device; determining an assessment module of the set of assessment modules that is associated with the location; and providing the assessment module.

### Example 19

The method of Example 18, further comprising determining the location using a global positioning device of the audit device.

### Example 20

The method of any one or more of Examples 11 through 19, further comprising determining a post-control risk score for each risk of the set of risks based on a severity score associated with each risk and a post-control occurrence score associated with each risk; calculating a total post-occurrence risk score for the audit site based upon the set of risks and the post-control risk score for each risk; and displaying the total post-occurrence risk score.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Other types of instruments into which the teachings herein may be incorporated will be apparent to those of ordinary skill in the art.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

1. An audit device comprising a processor configured to execute instructions stored on a memory, a display operable by the processor to provide information to a user, and a user input device operable by the user to provide input to the processor, wherein the processor is configured to:
provide a set of assessment modules via the display, wherein each of the set of assessment modules describes one or more observable risk indicators of an audit site;
receive a set of responses, wherein each response is associated with an assessment module of the set of assessment module and indicates whether an observable risk indicator is present at the audit site;
determine a set of risks based upon the set of responses, wherein each risk of the set of risks corresponds with at least one observable risk indicator within the set of responses;
calculate a total risk score for the audit site based upon the set of risks; and
display the total risk score.

2. The audit device of claim 1, wherein the set of risks is determined from a risk database including at least seven hundred risks, and wherein each risk in the set of risks includes a severity score and an occurrence score, and wherein the processor is configured to calculate the total risk score based upon the severity score and the occurrence score of each risk.

3. The audit device of claim 2, wherein each risk of the set of risks is associated with a capability of the audit site, and wherein the processor is configured to calculate and display a capability total risk score for each capability based upon the set of risks.

4. The audit device of claim 3, wherein each capability is within a set of capabilities including radiology, transfer to pathology, grossing, fixation, embedding, monotomy, staining, coverslipping, slide preparation, and immunohistochemistry.

5. The audit device of claim 3, wherein the processor is further configured to display the set of capability total risk scores sorted from highest to lowest.

6. The audit device of claim 1, further comprising an image capture device, wherein the processor is further configured to:
determine that an assessment module of the set of assessment modules is configured to request an observable risk indicator image;
when providing the assessment module via the display, provide an image capture interface operable to capture an image of that observable risk indicator with the image capture device; and
associate the image with a response for the assessment module.

7. The audit device of claim 1, further comprising a step counting device, wherein the processor is further configured to:
determine that an assessment module of the set of assessment modules is configured to request a distance measurement;
when providing the assessment module via the display, provide a distance measurement interface operable to measure a distance between a start location and a destination location using the step counting device; and
associate the distance with a response for the assessment module.

8. The audit device of claim 1, wherein the processor is further configured to, when providing the set of assessment modules:
determine a location of the audit device; determine an assessment module of the set of assessment modules that is associated with the location; and provide the assessment module.

9. The audit device of claim 8, further comprising a global positioning device, wherein the processor is further configured to determine the location using the global positioning device.

10. The audit device of claim 1, wherein the processor is further configured to:
associate a post-control risk score with each risk of the set of risks based on a severity score associated with each risk and a post-control occurrence score associated with each risk;
calculate a total post-occurrence risk score for the audit site based upon the set of risks and the post-control risk score for each risk; and
display the total post-occurrence risk score.

11. A method for providing a guided assessment of risk at an audit site comprising:
(a) providing a set of assessment modules via a display of an audit device, wherein each of the set of assessment modules describes one or more observable risk indicators of an audit site;
(b) receiving a set of responses via a user input device of the audit device, wherein each response is associated with an assessment module of the set of assessment module and indicates whether an observable risk indicator is present at the audit site;
(c) determining a set of risks based upon the set of responses, wherein each risk of the set of risks corresponds with at least one observable risk indicator within the set of responses;
(d) calculating a total risk score for the audit site based upon the set of risks; and
(e) displaying the total risk score.

12. The method of claim 11, wherein the set of risks is determined from a risk database including at least seven hundred risks, and wherein each risk in the set of risks includes a severity score and an occurrence score,
the method further comprising calculating the total risk score based upon the severity score and the occurrence score of each risk.

13. The method of claim 12, wherein each risk of the set of risks is associated with a capability of the audit site, the method further comprising calculating and displaying a capability total risk score for each capability based upon the set of risks.

14. The method of claim 13, wherein each capability is within a set of capabilities including radiology, transfer to pathology, grossing, fixation, embedding, monotomy, staining, coverslipping, slide preparation, and immunohistochemistry.

15. The method of any one or more of claim 13, further comprising displaying the set of capability total risk scores sorted from highest to lowest.

16. The method of claim 11, further comprising:
determining that an assessment module of the set of assessment modules is configured to request an observable risk indicator image;
when providing the assessment module via the display, providing an image capture interface operable to capture an image of that observable risk indicator with an image capture device of the audit device; and
associating the image with a response for the assessment module.

17. The method of claim 11, further comprising: determining that an assessment module of the set of assessment modules is configured to request a distance measurement;
when providing the assessment module via the display, providing a distance measurement interface operable to measure a distance between a start location and a destination location using a step counting device of the audit device; and
associating the distance with a response for the assessment module.

18. The method of claim 11, further comprising, when providing the set of assessment modules:
determining a location of the audit device;
determining an assessment module of the set of assessment modules that is associated with the location; and
providing the assessment module.

19. The method of claim 18, further comprising determining the location using a global positioning device of the audit device.

20. The method of claim 11, further comprising
determining a post-control risk score for each risk of the set of risks based on a severity score associated with each risk and a post-control occurrence score associated with each risk;
calculating a total post-occurrence risk score for the audit site based upon the set of risks and the post-control risk score for each risk; and displaying the total post-occurrence risk score.
